(19) United States
(12) Patent Application Publication
(10) Pub. No.: US 2022/0202757 A1
(43) Pub. Date: Jun. 30, 2022

(54) PROTEIN SUBSTITUTE AMINO ACID COMPOSITIONS AND PRODUCTS, AND OTHER FOOD AND BEVERAGE PRODUCTS

(71) Applicant: Martin Munzer, Boca Raton, FL (US)

(72) Inventor: Martin Munzer, Boca Raton, FL (US)

(21) Appl. No.: 17/601,976

(22) PCT Filed: Apr. 9, 2020
(86) PCT No.: PCT/US2020/027549

§ 371 Date: Oct. 7, 2021

Related U.S. Application Data
(60) Provisional application No. 62/831,690, filed on Apr. 9, 2019.

(51) Int. Cl. A61K 31/198; A61K 45/06; A23L 2/52

(52) U.S. Cl. CPC A61K 31/198; A23L 2/52; A61K 45/06

(57) ABSTRACT

Protein substitute products that furnish nutrients include amino acid compositions that include essential amino acids and non-essential amino acids in a preferred ratio, along with Vitamins, nutrients and botanicals provided as a food or beverage product that provides sustenance. Amino acids are provided in ratios and with other components to stimulate production of mitochondria in the cells and increase mitochondrial density in each cell type, including in particular muscle cells. Compositions may include simple and complex sugar producing carbohydrates to provide a short to medium energy source to the production of ATP by the mitochondria.

### BACKGROUND OF THE INVENTION

### 1. Field of the Invention

The present invention relates to the field of nutrition and more particularly to amino acid compositions for substitution or enhancement of protein, and more particularly to compositions for an animal and plant protein substitute in foods. Food and beverage products that provide amino acid components and other compounds to deliver a synergistic effect also are provided.

### 2. Background of the Invention

Protein is a macronutrient and is the primary source of life critical nutrients to humans and animals. It is one of three sources, next to fat and carbohydrates used by mammals to produce energy. However, unlike fat and carbohydrates, the primary components of protein, namely amino acids are essential for the production of metabolites such as those that build muscle, skin, collagen, bones, enzymes and hormones needed for the body to function properly. As our planetary population increases and climatic changes impact an already stressed global food supply, methods must be implemented to produce life critical amino acid nutrient supplies that are not vulnerable to weather, population growth or other factors influencing the quality and quantity of our global food supply. The object of this invention is to substitute animal or plant produced protein with what life forms ultimately derive from proteins, namely amino acids. When derived from animals or plants, approximately 50% or more of the proteins metabolized are essential only for the host that provides the protein, but not to the one who consumes the protein. These non-essential amino acids require the same expenditure of digestive energy to break them down as those that are useful for the consuming person. Compounding this digestive energy expenditure is the fact that they then also require metabolic energy to eliminate and detoxify them from the body. The Environmental Working Group (http://www.ewg.org/meateatersguide/at-a-glance-brochure/) calculated that if everyone in the U.S.A. ate no meat or cheese just one day a week, the environmental impact would be that of cars driving 91 billion miles less, or taking 7.6 million cars off the road. The impact of replacing one meal a day, all week long with a supplement that is actually a healthy alternative to a conventional meal, and helps build muscle, sharpen the mind and helps achieve a higher state of wellness could have a significant impact on the environment and economy as a whole. It also can serve to relieve the stress to the food supply issues being experienced globally due to a growing population in an unstable weather environment. A number of foods and consumable products are generated today through processing of plant-based as well as animal-based materials. In some instances foods may not be tolerated by particular individuals, but a way to obtain the nutritional value or equivalent is desired. There are food substitutes that have been developed, including for example, food substitutes to take the place of meat, where, for example, plant-based substrates are utilized and are processed to replicate the taste and even texture of meat, although the product contains no meat. The Impossible Burger® by Impossible Foods Inc. is one example of a plant-based meat substitute. The product seeks to recreate a realistic beef taste, which some report to be achieved through a compound called “heme”. It has been reported that the company is able to extract heme from specific plants, and then ferment the heme in a process that is similar to how the Belgians have been making beer for nearly a thousand years. (See www.digitaltrends.com/features/the-future-of-food-meat-alternatives) Myoglobin gives muscle its characteristic taste, and soybean roots contain leghemoglobin, a protein whose 3-D structure is very similar to that of myoglobin. Impossible Foods “takes genes which code for the soy leghemoglobin protein and inserts them into a species of yeast called Pichia pastoris.” The yeast, with some “modified yeast sugar” and minerals, replicate heme efficiently. Often substitutes have beneficial effects, such as conservation of resources. For example, compared weight to weight, an Impossible Burger may use 1/20 of the land and ¼ of the water required for production of a normal beef patty, while producing ⅛ of the greenhouse gases. (See www.wired.com/story/the-impossible-burger) From a beef patty, when myoglobin is cooked, it unfolds and exposes the heme cofactor (prosthetic group). The heme catalyzes reactions which transform amino acids and other, natural, organic compounds into flavor and aroma compounds. Leghemoglobin accomplishes this task because it unfolds similarly, and thus results in a similar flavor as that found in cooked meat. Leghemoglobin does have a primary amino acid sequence which diverges from those in animal myoglobins, but the 3-D structure is very similar, as mentioned before, which leads it to behave in a similar manner, when cooked, to myoglobin.

Hemoproteins are proteins which have heme as their prosthetic group (the non-protein compound which is required for an enzyme's activity as a catalyst).

An example of a non-meat consumable is disclosed in U.S. Pat. No. 10,172,381, assigned to Impossible Foods Inc. Other patents also relate to production of food products include U.S. Pat. Nos. 9,943,096; 9,938,327; and 9,808,029.

US patent application US 2019/0133163 A1 discloses “Ground Meat Replicas” and methods of making the replicas using an isolated plant protein in addition to a heme-containing protein. The method is disclosed to result in a “meat dough” product that the '163 patent application considers suitable for use in place of ground meat. The '163 patent application refers to a flavor precursor which is disclosed to contain at least one amino acid.

US patent application US 2017/0105438 A1, assigned to Savage River, Inc. dba Beyond Meat, discloses “Meat-Like Food Products” that are disclosed to be meat substitute products which may serve as substitutes for animal meat. The '438 application discloses that the products may be made from a mixture of various flours, carbohydrates, fibers, and other ingredients, and that it may contain any one of a number of numerous other ingredients, from about 0.01% to about 5%, among which include including amino acids and amino acid derivatives.

International patent application WO 2017/014654 A1, assigned to Kubura, Spólka Jawna, discloses a vegetable-based meat substitute that is made from a germinated leguminous seeds as well as flour from leguminous plants. The seeds are soaked in a solution with additives (e.g. vitamins, minerals, or salts, to name a few) during while they are germinating so that the additives are absorbed by the seeds. Exogenous amino acids may be added to the meat substitute. A purpose disclosed in this patent application for this meat substitute is that is can be used entirely in place of meat, since the application considers that the product contains proteins and nutritive substance equivalents without any ingredients from animal origin (p. 2, ¶3).

International patent application WO 2017/153931 A1, assigned to Tereos Starch & Sweeteners Belgium, discloses a moist pet food composition comprised of a meat analogue disclosed as a vegetable protein. The '931 application considers the product disclosed to improve digestion or periodontal health in a companion animal (i.e., pet). This pet food may be comprised of two components: a water-based component and a solid component that comprises a meat analogue. In the mixing step of the meat analogue, “food additives”, such as, for example, simulated meat flavorings, savory flavorings, minerals, amino acids, and vitamins, and other components, may be added.

US patent application US 2003/0186417 A1, assigned to Danisco A/S, discloses an amino acid sequence for use in meat substitutes. The '417 meat substitute is pectin-based. Pectin contains galacturonic acids and residues, and when the carboxyl groups of those residues are methylated, the degree to which they are methylated determines whether the pectin is classified as an HE (high ester pectin) or an LE (low ester pectin). This is significant, because the structure of the pectin, (the degree of esterification (methylation)) determines the resultant chemical and physical properties of the pectin. Pectin Methylesterases (PMEs) de-esterify HEs and LEs. Some PMEs are found naturally in different plants (for example, orange, apple, apricot, bean, carrot, and radish).

European patent application EP 20140179883, assigned to Stichting, Eco Consult, discloses a method for production of a meat substitute for vegetarian consumption, using solid state fermentation. The '883 application discloses mushroom mycelium added to a substrate which can contain shrimp shells, seaweed, algae, and/or fish residues. The mushroom mycelium grows in the substrate, and the result is a meat substitute composition. The composition may also contain other ingredients like sawdust, apple peelings, and asparagus peelings, to name a few. The disclosure makes reference to meeting requirements for meat substitutes, such as: having a meat-like texture, containing essential amino acids, and providing a desired taste profile.

A meat substitute also is disclosed in US patent application US 2018/0303125, assigned to Valio Ltd (Helsinki, FI), that provides a casein-containing material. The resulting product is disclosed to be used as a cheese or meat substitute product, suitable for frying or grilling. Casein is the main protein found in milk and cheese, and contains all of the essential amino acids, which the '125 application references. The '125 patent application specifies a cross-linking enzyme, transglutaminase, which is disclosed for the purpose of catalyzing the generation of covalent linkages between the glutamine and lysine amino acid residues present in the protein molecules. Because caseins, especially β-casein, are considered to be rich in lysine and glutamine, they are disclosed as the best substrates for transglutaminase.

International Patent applications WO 2018/115595 and WO 2018/115594 also assigned to Valio Ltd, disclose meat substitutes suitable for microwaving, frying, or grilling; however, WO 2018/115595 describes a plant-based protein product while WO 2018/115594 describes a milk protein product, in which the exemplary composition is crumbled cheese. Both of these latter Valio Ltd patent applications disclose an additional, optional process steps which may include adding additional ingredients such as amino acids.

International patent application WO2018/177717, assigned to Unilever N.V., discloses a method for preparing a meat analogue for use as a generic meat substitute for human consumption. Xanthan gum and galactomannan are used as gelling agents, and when the meat analogue is cooked, it is disclosed that the gel matrix melts and releases liquid, similar to juice released from cooking meat.

US patent application 2012/0315365, assigned to Solae, LLC, discloses a meat composition which contains n-3 PUFAs (polyunsaturated fatty acids). The primary composition is actual animal meat, and the '365 patent application discloses “o-, m- or p-amino benzoic acid (o is anthranilic acid, p is PABA)” as additional ingredients for the composition.

A need exists for a product that may provide nutrition in a manner that is beneficially available for use to supply the nutritional needs of an individual and which may be a replacement for or supplement for a natural meat product.

### SUMMARY OF THE INVENTION

Nutritional compositions comprised of amino acids are provided. According to preferred embodiments, the amino acid compositions are provided as a substitute for meat, vegetable and other protein products. Compositions include amino acid compositions for substitution or enhancement and to produce a protein substitute, such as a substitute meat product, or other food. For example, the nutritional compositions may be consumed in the form of food or nutritional ready-to-drink beverage. Further ingredients may be included as needed for optimal flavor and texture choices, and to prevent spoilage.

Embodiments of the compositions of the invention may include food or beverage products that may provide a substitute for meat or other dietary protein sources. The food or beverage products preferably include the nutritional compositions, including amino acids, and may be composed with binders or other components to form a food product or beverage.

Alternatively, the food and beverage products may be provided as a supplement that may be added to existing food or beverage products.

The food and beverage products may also comprise replicas of food products, such as, for example, meat replicas, which may be in the form of a meat patty. Food replicas may comprise replicas of poultry, fish and other food products. The food and beverage products, including the food replicas, preferably are produced with the amino acid formulations according to the invention, which may include amino acid ratios and compositions, according to the ratios set forth herein. According to some embodiments, the food and beverage compositions, including the food replicas, contain the amino acid compositions and also include other micro- and or macro-nutrients as well as botanical ingredients with functional qualities, such as a cell protective, anti-inflammatory or immune system modulating compound and a neuro-regenerative compound which are provided as components of the food and beverage products and/or food replicas.

According to some embodiments, the compositions of the invention may be provided in dosages or metered amounts, such that for example, each dosage is supplied or packaged in a readily usable amount or dosage form, wherein each dosage form of the composition may be a dosage form of an ingestible format (such as a food or beverage) wherein the food or beverage component, includes a pre-selected volume of the ingredients of the composition (e.g., such as a pre-selected amount of total ingredients, of active ingredients, and/or of amino acids or amino acid ratioed components), as set out in the preferred embodiments of the composition mixtures and formulations herein. The term “dosage form” as used herein is intended as an all-inclusive term that contemplates that the food of the present invention is packaged and/or supplied in any one or more of a number of forms having a volume that provides a prescribed amount of the ingredients set out in the formulations herein for consumption by an individual for the purpose of obtaining the nutrient values from the ingredients, and the composition synergies of the ratioed and combined components of the formulation, to provide general nutrients, or to address a potential lack of a dietary nutrient due to a deficiency, a geographic regional unavailability, or bio-unavailability from typical sources, or other need for supplementation or replacement. The compositions preferably are formulated to deliver nutrients which preferably comprise amino acids, as well as vitamins, minerals, fats, carbohydrates and botanicals, which can be ingested as a food or beverage product, and/or for the purpose of a desired range of the composition components. Preferably, the compositions are supplied in a convenient and pleasant tasting form.

Some examples of the present compositions include providing the composition in the form of a food bar, or nutrition bar, preferably supplied in poly-foil or other types of wrappers, as is commonly seen in most food markets, convenience stores and health food stores. For example, the food or nutrition bars may be produced by any suitable method including a machine extrusion process that extrudes the mixed ingredients into the desired size and shape bar, which may be packaged by conveyance delivery to an automatic wrapping apparatus.

According to some alternate embodiments, the compositions may be supplied in a baked or cooked food product or snack; while in alternate embodiments, the food product may be provided as a frozen, dried, vacuum packaged, or other form for storage and use.

According to preferred embodiments, the food products produced and formed with the compositions herein preferably are provided in a pleasant tasting and in a convenient form for ready use or mixing. The food products, with or without the amino acids, vitamins, minerals, and/or other nutritional supplements compositions already contained therein, may also be extruded, baked, rolled, pressed, cut or otherwise formed into bars, cookies, brownies, cakes or muffins. In the manufacturing process for bars that are extruded, other ingredients may be used to bind, carry or deliver the nutritional compositions of the invention. Known processes may be used to form food products of the invention that include the nutritional compositions and components of the invention.

According to yet other embodiments, the nutritional composition comprises a ready-to-drink beverage, requiring no addition of water and/or mixing with water or other liquids. For example, the composition may be supplied in a ready to drink form, fully constituted, with no additions required; or alternatively, the compositions of the invention may comprise a powder or a liquid concentrate that is provided to be mixed with water, fruit juice, fruit and/or other flavored drinks, and/or fruit drink concentrates to make, for instance, a flavored beverage. According to alternative embodiments, the composition may be provided in a mixture that may be added to or mixed with milk to make a drink that is similar to the properties of a “milk shake”. Embodiments may comprise pre-measured amounts of compositions containing pre dosed amounts of the nutrients, such as the amino acids and/or ratioed amino acids, vitamins, minerals, botanicals and/or other components, as described hereon, which may be in the form of a measured amount of a powder, in a packet or container (which may be a single size for addition to a 12 oz or 16 oz juice or water bottle, or the like). For example, the packaged composition may be supplied for mixing with water, fruit juice, flavored beverage, and/or milk to provide a suitable or recommended dosage amount of the nutrients of the present invention.

The present invention also may be provided with suitable flavorants and/or masking agents, to provide a pleasant taste, for use whether supplied in a constituted form, such as a beverage, bar or other snack form, or provided alternatively, as an additive to be added to water, juice or food. The products provided may be used at home, or while away from home or participating in an activity or traveling. The product may be provided in packing that is compatible with travel restrictions, and may be subsequently mixed with readily available foods and beverages, including potential health benefits of ingesting a food bar or other such product containing the nutrients, or as a powder to be mixed with a liquid, and in the ready-to-drink beverage mode.

According to some embodiments, the compositions may be embodied in a food or may be mixed with other substances or liquids either at the time of consumption or manufacture. For example, according to some embodiments, the compositions may be provided in a form mixed with liquids, such as where it is packaged and sold in an appropriate convenience-type container, or packaged as a liquid in a volume that provides a dosage amount of the prescribed volume, with the prescribed nutrient amounts. According to some other embodiments, a powder may be manufactured in accordance with the present invention and may be provided in a form suitable for addition to foods, for example, such as sauces and soups for consumption as part of a normal meal, with the preferred nutrient volumes present in the dosage amount added to the foods.

The compositions, which may be embodied in food and/or beverage products of the present invention in some embodiments provide complete or near complete nutritional requirements, and in other embodiments provide supplementation of dietary nutrients, through compositions and their administration. The products of the invention are formulated to provide bioavailable nutrients that may be taken up by the individual. According to some implementations, a regular diet of the food or beverage product may provide continued nutrients. According to some implementations, the products may contain prescribed dosages of the nutrients, Vitamins, minerals and other ingredients, such as botanicals, including the content of one or more the aforementioned being provided in amounts or ratios to provide a synergistic benefit. The compositions are designed to provide nourishment, and preferably that is bioavailable, and is provided with compatible components that are formulated together to make use of the available nutrients, including proteins, minerals, and Vitamins, as well as botanical substances, that aid in metabolic and regenerative pathways.

According to preferred embodiments, the products of the invention preferably comprise an amino acid mixture. Some preferred embodiments also provide the amino acid mixture along with one or more Vitamins, and preferably Vitamin combinations that provide benefits when included together with other Vitamins, and together with the amino acids.

According to some preferred embodiments, the amino acid compositions may include, for example, Vitamin K2 (preferably Vitamin K2 MK7). The mixture also may include one or more of a cell protecting component, epigenetic and a neuro-regenerative component. The compositions may be formulated with additional ingredients, where embodiments of the composition may include additional Vitamins, minerals and/or botanicals. Inactive ingredients may be included as well, such as for example, flavorings, masking, thickening and binding compounds. According to preferred embodiments, the compositions include Vitamin B3. Vitamin B3 (niacin), preferably is provided in the form of niacinamide. High levels of Vitamin B3 may be beneficial to provide effective benefits, including, for example, as a cholesterol regulator to boost the levels of good HDL and to lower triglycerides. However, the problem with high doses is that despite the benefit of cholesterol regulation, at higher doses, it has been reported to have detrimental effects on the liver, cause prostate and gastrointestinal problems and/or glucose intolerance. The present composition provides a combination of amino acids along with Vitamin B3 (niacinamide), to provide effective synergistic benefits of the Vitamin B3 without the potential harm of the high dosages. The compositions preferably include less than the maximum 35 mg maximum daily intake, and preferably provide about 125% of the recommended daily allowance, or 20 mg per serving size of the composition. The composition provides for synergistic usage improvement of the Vitamin B3. The present compositions provide nutrients, such as for example, amino acids, and provide metabolic enhancing components, such as the botanicals and minerals, to facilitate usage of the amino acids. According to preferred embodiments, the amino acids are provided to supply the building blocks of proteins from which to build or regenerate various tissues and cells. In the present compositions, the Vitamin B3 is present with the amino acid composition, in the amounts provided by the formulations, to facilitate neural health, and promote regeneration and/or myelination.

According to preferred compositions, the cell protective component may comprise phosphatidylserine (70% as SerinAid®), and the neuro-regenerative component may comprise Vitamin B3 (niacinamide).

Compositions of the invention preferably also include one or more substances that promote calcium and magnesium metabolization, and facilitate DNA repair and/or protein synthesis, with a preferred substance comprising boron. The compositions may include boron, which may be provided in the form of a boron-containing compound, such as, for example, according to preferred compositions, boric acid, and alternatively, or in addition, boron citrate, boron glycinate, and/or boron aspartate. The boron, such as, for example, boric acid, is provided in concert with an amount of Vitamin K2, and preferably Vitamin K2 MK7 form. According to preferred compositions the boron and Vitamin K2 contribute to a synergistic effect, and together with one or more other components of the compositions, such as the amino acid blend, provide a calcium regulation feature. Food and beverage compositions according to the invention may include boron and Vitamin K2, and preferably the MK7 form. For example, due to a disease or injury, or lengths of time where the individual does not engage in activities that put weight on the bone, the tendency is for the bone to demineralize itself of calcium. The calcium may build in arteries, as well as pass through the kidneys where stones may form. According to some embodiments, the present food and beverage compositions may include Vitamin K2 MK7 in an amount that is major relative to the minor amount of boric acid. According to some preferred compositions, the boric acid is provided in an amount less than 50% of the amount of Vitamin K2 MK7 (MenaQ7 Pure 2000), and, preferably about 30 to 50% of the weight or amount of Vitamin K2 MK7 (MenaQ7 Pure 2000). The Vitamin K2 MK7 (MenaQ7 Pure 2000) preferably is provided in an amount of about 0.5% to 0.9% active ingredients, by weight, and preferably the boric acid is provided in an amount of about 0.2% to about 0.5%, and preferably keeping within the preferred ratio of about 30 to 50% of the Vitamin K2 MK7.

According to some preferred implementations and usage, the food and beverage products may be formulated as a total nutritional product, or may be provided as a nutritional product that can be taken with or supplemented with other products (e.g., other foods and beverages. According to some embodiments, the food and beverage products may be taken, e.g., via ingestion, along with a carbohydrate (preferably complex carbohydrates), and preferably also with a fat. According to some other embodiments, the carbohydrate and fat are formulated as part of the food or beverage product.

Some examples of carbohydrates with which the compositions of the invention may be taken or which may be formulated as part of the food or beverage composition, include, whole grain bread, oatmeal (e.g., old-fashioned or steel cut) and other multigrain cereals (e.g., mix or barley, tapioca, oats, rye, triticale), yams (sweet potatoes), brown rice, basmati), quinoa, couscous, pumpkin, butternut squash, and beets. Additionally, preferably the compositions also are taken along with a fat (such as an edible oil). Some examples of fats include oils from botanical nuts or tree nuts, such as coconut oil. Preferred embodiments of compositions may include a carbohydrate, and/or a fat, and more preferably, both carbohydrate and fat. Some examples of complex carbohydrates that may be included as part of the composition include maltodextrin, and some examples of fats include lecithin (e.g., such as, for example, sunflower or canola lecithin). Other complex carbohydrates may be used with or as a vehicle to deliver the compositions of the invention (e.g., amino acid mixture, Vitamins, and other components).

According to preferred embodiments, the food and beverage compositions of the invention contain amino acids in free form, thus they are absorbed without using digestive energy in a highly efficient manner (which may be up to nearly 100% absorption). Based on equivalency of what can be derived from protein, the recommended daily consumption of two servings of the present compositions provides approximately ⅔ of the amino acids that an average weight adult (140-150 lbs) needs each day. Embodiments of the present compositions also may include other key vitamins, minerals and botanical ingredients which provide building blocks for various metabolic cycles and epigenetic regulation that are required for the optimal function of a person's metabolism. The present compositions preferably comprise nutrients that are in a bio-available form, with many nutrients being in the most bio-available viable form for the best uptake by the body.

Preferred compositions are formulated to include amino acids and Vitamins, and further include one or more, and preferably both, a cell protective compound and a neuro-regenerative compound. A synergistic effect is realized by combining one or more of the cell protective compound and neuro-regenerative compound, and preferably both, together with the amino acid blend. The food and beverage products preferably include the amino acids and Vitamins, and further include one or more, and preferably both, a cell protective compound and a neuro-regenerative compound, and may include additional compounds to serve as a binder or filler for forming the components into a ready to eat or ready to use food or beverage product.

According to some embodiments, some of the ingredients of the current formulation may themselves be patented substances (which may be procured or produced by license, or produced according to a patent (in force or expired): (Curcumin C3 Complex: U.S. Pat. No. 5,861,415 and EP0839037, SerinAid®: U.S. Pat. No. 8,124,594, MenaQ7®: U.S. Pat. No. 9,828,323). The composition is formulated with ingredients, many of which are obtained and are based on the unique manner that they are extracted, prepared or their biochemical characteristics. According to a preferred embodiment of a composition, up to 8 ingredients may be specially procured, and preferably there are 11 ingredients in the composition that are extracted, obtained or otherwise prepared in a manner that provides improved or maximized bio-availability.

According to preferred embodiments, the present compositions may contain Vitamin B9, which may be provided through folic acid component of the present composition. However, the present composition, unlike common folic acid ingredients that 1 of 3 individuals could not make proper use of, contains a bio-available form of folic acid, enabling those who are unable to convert the folic acid to the needed Vitamin B9 to benefit.

The compositions of the present invention are formulated with ingredients that are classified as being safe for consumption of the compositions on a daily basis. The present compositions contain essential vitamins, minerals, amino acids and specific botanical substances and/or their extracts. The components of the present compositions preferably comprise products that are clinically proven to be the building blocks and promoters that impact genetic function in a variety of ways. The ingredients of the present compositions have been selected and formulated to synergistically, and in a positive way, impact DNA methylation, histone acetylation or de-acetylation, DNA repair, gene expression, telomere maintenance and stem cell production.

The present composition formulation embodiments incorporate a considerable number of ingredients that are involved in the process of DNA repair and/or protection from DNA damage. These properties are critical towards achieving and maintaining an optimal state of wellness. DNA repair is a natural mechanism directed at fixing DNA damage caused by free-radicals, environmental influences or other means. Over 2,400 human clinical studies on individual ingredients of the present compositions have demonstrated that they have properties to positively impact DNA.

The present compositions are provided to be taken in a dosage regularly, preferably daily over the course of a few weeks or months. The dosage preferably may depend on the condition of the individual, the weight of the individual, as well as other factors.

According to preferred embodiments, the present compositions are designed to reduce fatigue and provide increased energy throughout the day, as well as to improve sleep, concentration, and skin and muscle tone.

According to preferred embodiments the compositions are formulated with components designed to provide amounts of nutrients, in particular ratios and/or percentages, that synergistically contribute to usage of the nutrients by an individual's metabolism.

The present compositions are designed for use by adults and young adults, and are provided for maintenance of a high degree of wellness and a strong physique, including as they age. The present compositions also are provided to benefit those who have reached an advanced age and desire to use nutrient supplementation to reduce or potentially minimize their frailty and lack of energy. The present compositions also may benefit individuals that need to recover from a weakened state, regardless of the cause. The present compositions preferably provide highly bioavailable ingredients known to be essential building blocks, needed to help the body restore and repair aspects of its physiology. Preferred embodiments of the formulations are produced from compounds that were produced without the use of hormones, antibiotics, pesticides, or contain contaminants, such as glyphosate and harmful heavy metals such as arsenic, mercury, lead or cadmium (as may otherwise be found in protein concentrates).

According to preferred embodiments, the present compositions are designed to not include any non-essential amino acids that the human body cannot utilize or metabolize, and that need to be detoxified by humans when co-consumed with any dietary protein sources. According to some preferred embodiments, preferably, the binders and fillers used to form the food and beverage products also are comprised of substances that will not introduce non-essential amino acids that the human body is not able to utilize or metabolize. The food and beverage products, according to preferred embodiments, avoid the use of the non-essential amino acids and/or substances that the human body, of a presumed normal functioning human, does not require or metabolize. According to some other preferred embodiments, the food and beverage products are formulated to avoid the use of the non-essential amino acids and/or substances that the human body, of a presumed normal functioning human, cannot utilize for metabolic or other functions.

In addition, the present food and beverage compositions formulated in accordance with the invention are designed to minimize the digestive energy required for their decomposition and absorption by the gastro-intestinal system. Protein sources that are consumed generally require that approximately one half of the energy contained in the protein, is required for their digestion and breakdown into the nutrients that the body can absorb.

The present compositions may have benefits, and are designed to increase the body's nitrogen balance, in order to achieve an anabolic state which means that the body is building metabolites and repairing tissue.

The present compositions are designed to help reduce existing inflammation in the body, thereby assisting in the tissue repair process.

The present compositions are designed to stimulate the re-growth and repair of neurons and their connections (dendrites, synapses and axons) to organs, nerve cells and muscles.

The present compositions are designed to stimulate the production of mitochondria in the cells, and increasing the mitochondrial density in each cell type, but especially in those of the muscle cells. Mitochondria are responsible for the conversion of nutrients into Adenosine Tri-Phosphate (ATP), which is the fuel used to produce energy in the body.

Preferred embodiments or the food and beverage products formed using the present compositions are designed to replace or reduce a need or perceived need for caffeine (e.g., caffeinated beverages, and potions), and instead of producing a mere effect (e.g., by stimulating the body), provide bioavailable nutrients for the body to build proteins and other metabolite compositions needed to supply energy to the body. The present compositions preferably may provide improvement for development and aging, as nutrients, in particular vitamins, may benefit individuals. See, e.g., Abstract from the 70th Anniversary Conference on ‘Vitamins in early development and healthy aging: impact on infectious and chronic disease’ 15-17, June 2011, American Society for Nutrition: “Nutrients Can Act as the Source of Epigenetic Modifications and Can Regulate the Placement of These (Methylation) Modifications.”

### DETAILED DESCRIPTION OF THE PREFERRED EMBODIMENTS

The present compositions include food and beverage compositions which preferably include an amino acid component, which, according to some preferred embodiments, comprises a mixture of amino acids, and more particularly a ratioed proportion of amino acids to provide synergistic effects. According to embodiments, vitamins, botanicals, minerals and other nutrients may be included to form the composition. Products also may include one or more of fats and/or carbohydrates, and mixtures of these components. According to some preferred embodiments, the food and beverage compositions may be ready to use products, or, according to alternate embodiments, may be provided to be used with other foods or beverages as an adjunct or supplemental product.

The ratioed amino acids preferably are included in the food and beverage product formulations to include essential amino acids (EAAs) and certain non-essential amino acids (non-EAAs), and according to some preferred embodiments, the food or beverage products contain EAAs selected from the group consisting of: isoleucine, leucine, valine, histidine, lysine, methionine, phenylalanine, threonine, and tryptophan; and also contain non-EAAs selected from the group consisting of: arginine, cysteine, cystine, glycine, proline, and tyrosine. In a preferred exemplary embodiment, the food and beverage compositions contain at least each of the preferred EAAs and non-EAAs, so that there is present in the food or beverage product each of: isoleucine, leucine, valine, histidine, lysine, methionine, phenylalanine, threonine, and tryptophan; and each of: arginine, cysteine, cystine, glycine, proline, and tyrosine.

According to some preferred formulations, the food or beverage products contain EAAs of about 74% to 77% by weight of the total amino acids of the product, and non-EAAs comprise about 23% to 26% by weight of the total amino acids of the product. The amino acids preferably may comprise the ratioed amino acids of the amino acid portion of the formulation. The amino acids preferably are added to comprise the food or beverage product as a nutritional fraction of the resultant product. The nutritional fraction may include one or more additional components, such as the Vitamins, minerals, botanicals, carbohydrates, and fats (and other components of the formulations disclosed and described herein. According to some embodiments, the food or beverage product contains only the amino acids of the nutritional fraction, and the binder or filler or other components used to form the food or beverage product do not introduce any additional amino acids or substantially no additional amino acids into the product. According to some other embodiments, where additional components forming the food or beverage product introduce yet additional amino acids into the product (for example, as part of the binder or filler or other component), the ratios may relate to the amino acids of the amino acids that are presented as part of the nutritional fraction of the food or beverage component. Alternatively, the ratios may be determined for the resultant food or beverage product, and the amino acids of the nutritional fraction may be adjusted to compensate for the potential addition or reduction of amino acids content that the formation process used to form the food product or beverage product may introduce, including for example, where the additional material, such as the binder and/or filler contributes to the presence or absence of the amino acids, and including where it may alter the preferred amino acid ratios.

According to preferred embodiments, the compositions may be provided via a fat substance, such as, for example, a nut butter or other natural product. In some instances, the amino acids may be added in the ratios provided to provide a minimum amount of amino acids, and the additional amino acids of the fat or nut butter may be present to contribute additionally to the amino acids of the nutritional fraction. The amino acid adjustment may be made to account for the amino acids, such as for example, considering that 100 g of peanut butter has about 1200 mg phenylalanine, and also contains histidine and methionine. According to a preferred amino acid formulation, histidine, methionine and phenylalanine, about 2.5 to 3% each of the amino acid content (with some preferred embodiments histidine being above 3% and each of methionine and phenylalanine being below 3%). Therefore, in order to maintain the preferred ratios, the amino acid nutritional fraction may be adjusted to account for the amino acids present in the additional products, such as the nut butter in this example.

A food substitute that provides nutritional value to a mammal and in particular a human, is provided. The food substitute, according to some embodiments, may supersede or supplement foods, and may be taken as an additional supplementary nutrient component. According to some other embodiments, the composition is provided as a food product, and may be configured to represent a food configuration, or may be provided in any one or more other forms, such as bars, agglomerates, a beverage, as well as frozen bars or containered products. The amino acid compositions may be provided as a substitute for meat protein, and, according to preferred embodiments, preferably also include a one or more other nutritional components, such as Vitamins, minerals, and/or botanicals. According to preferred embodiments, the compositions are provided in prescribed amounts, and may be provided in relative amounts or ratios for improvement of synergistic effects of one or more of the constituents of the composition.

According to some embodiments, the compositions may include an edible fibrous component, which according to some embodiments may be a plant based material, such as for example, one or more, or combinations of any of the following: plant fibers from carrot, bamboo, pea, broccoli, potato, sweet potato, corn, whole grains, alfalfa, kale, celery, celery root, tapioca, parsley, cabbage, zucchini, green beans, kidney beans, black beans, red beans, white beans, beets, cauliflower, nuts, apple skins, oats, wheat, or psyllium, or a mixture thereof. According to some other embodiments, the composition may further include fibrous components such as wheat gluten and protein isolates, In some embodiments, the edible fibrous component can be a solution-spun protein fiber (e.g., a solution-spun protein fiber containing a prolamin such as corn zein, pea prolamin, kafirin, secalin, hordein, avenin, or a mixture thereof).

According to some embodiments, the compositions may further include a carbohydrate. The carbohydrate may comprise a suitable carbohydrate or mixture. Some examples of a carbohydrate may include a carbohydrate-based gel that may be formed from one or more materials comprising agar, pectin, carrageenan, konjac, alginate, chemically-modified agarose, or mixtures thereof. According to some other embodiments the carbohydrate may include a complex carbohydrate, such as, for example, maltodextrin.

Some embodiments of the nutritional food and beverage compositions according to the invention may include a fat. The fat may be provided in the form of an animal fat, or non-animal type fat, or mixtures of both types, non-animal based and animal based fats (or precursors). Some examples of fats include those which may be provided in the form of an edible oil. Some examples of fats that may be used to form some of the compositions of the invention include for example, an algal oil, a fungal oil, corn oil, olive oil, soy oil, peanut oil, walnut oil, almond oil, sesame oil, cottonseed oil, rapeseed oil, canola oil, safflower oil, sunflower oil, flax seed oil, hemp oil, avocado oil, palm oil, palm kernel oil, coconut oil, babassu oil, shea butter, mango butter, cocoa butter, wheat germ oil, borage oil, black currant oil, sea-buckhorn oil, macadamia oil, saw palmetto oil, conjugated linoleic oil, arachidonic acid enriched oil, docosahexaenoic acid (DHA) enriched oil, eicosapentaenoic acid (EPA) enriched oil, palm stearic acid, sea-buckhorn berry oil, macadamia oil, saw palmetto oil, or rice bran oil; or margarine or other hydrogenated fats. The fat may be provided in a form that includes one or more other components, such as for example, a flavoring agent, Vitamins, minerals and/or botanicals. For example, according to some compositions the fat soluble Vitamins may be provided as part of the fat addition to form the food product of beverage product. For example, the fat may be provided as a plant-based source of eicosapentaenoic acid (EPA) and docosahexaenoic acid (DHA), two omega-3 fats that benefit health. One example of a fat that also provides the omega-3 fats is algal oil. According to some embodiments, the fat can contain the flavoring agent.

As discussed herein, the compositions preferably include masking and/or flavoring agents. Some examples of a flavoring agent includes vegetable extracts, fruit extracts, acids, antioxidants, carotenoids, lactones, and combinations thereof. According to some embodiments, the antioxidant may be epigallocatechin gallate. According to some embodiments carotenes (e.g., β-carotene, α-carotene, lycopene) and xanthophylls (e.g., β-cryptoxanthin, lutein, zeaxanthin, canthaxanthin), or mixtures thereof may be provided as a flavoring agent as well as a nutrient. According to some examples, the vegetable extract can be from a cucumber or tomato. According to some examples, the fruit extract can be from a melon or pineapple.

The compositions may include a binding or forming agent to facilitate forming the nutrients such as the amino acids, as well as other components that may be included, such as, Vitamins, minerals and botanicals, and other components of the compositions disclosed and described herein. These mixtures may comprise or be formed into food product formatives or beverages. According to preferred embodiments, the binding agent may be a suitable component that is compatible with the nutrient and other components that provide the nutritional benefit. The binding agent may or may not add to the nutritional value. According to some embodiments, the binding agent or filler may comprise a fat, such as a nut product, that may preferably be used in a nut butter form. According to some embodiments, the binding agent may comprise an isolated plant protein. According to some embodiments, the binding agent may comprise milk or dark chocolate. According to some other embodiments, the binding agent may comprise a carbohydrate product, such as for example, a carbohydrate-based gel. Some examples of binding agents include products comprised from or consist of suitable plant proteins. Some examples of plant proteins include RuBisCO, albumin, gluten, glycinin, conglycinin, legumin, globulin, vicilin, conalbumin, gliadin, glutelin, glutenin, hordein, prolamin, phaseolin, proteinoplast, secalin, triticeae gluten, zein, oleosin, caloleosin, steroleosin, or mixtures thereof (e.g., albumin fractions). Plant proteins may be obtained from any suitable source, including for example, soy, peas or lentils, and/or other leguminous plants. According to some embodiments, binding agents may be obtained from a non-plant-based source, such as, for example, egg albumin or collagen.

In embodiments where the carbohydrate is supplied in the form of a carbohydrate gel, which can for example, be a mixture of simple and complex sugar producing carbohydrates such as from coconut palm sugar and fructose in addition to maltodextrin and resistant starches such as green banana flower or chicory root. The composition can be used to provide a short to medium energy source to the production of ATP by the mitochondria.

In most embodiments, the resulting composition can be supplied at room temperature so as to avoid the denaturation of the amino acids. However, heating may be desired to provide a more meal like feel to the resulting product. In these compositions the choice of amino acids used should reduce or exclude those that denature at higher temperatures between 120-350° F. and do not reconstitute efficiently or at all. These amino acids may be substituted or reinforced with conventional hydrolyzed protein sources that provide a favorable ratio of the amino acids.

When the binding agent is a protein, it is useful for the denaturation temperature of the protein to be less than the melting temperature of the carbohydrate-based gel. For example, the denaturation temperature of suitable protein-binding agents (e.g., RuBisCO, albumin, soybean conglycinin, or a gluten, or mixtures thereof) can be between about 40° C. and about 80° C. This allows the carbohydrate based gel to melt after the protein binding agent denatures and binds the meat replica together, and provides better texture and form to the meat replica.

According to some embodiments, a meat substitute is provided, and the nutrient compositions are processed with a binding component to produce a food product, which may be configured to comprise a food replica, such as for example, a meat replica. According to some embodiments, the amino acids, as well as one or more other components of the nutritional compositions (such as Vitamins, minerals and/or botanicals), may be provided in a vehicle that includes the binding agent. According to some embodiments, the binding agent may be any suitable compound or components that the amino acid composition, as well as any additional components, such as Vitamins, minerals, botanicals, fats, carbohydrates, according to the formulations herein, may be mixed with or combined to produce a stable food or beverage product.

According to some embodiments, the binding agent typically is selected from compounds that do not increase or remove the amino acids and the amino acid ratios. According to some other embodiments, the binder is accounted for in the formulation, such that the binder contribution to one or more of the formulated components (amino acids, Vitamins, (e.g., niacin)), is counted as part of the formula amount. For example, where the binder contains a carbohydrate source, then the maltodextrin carbohydrate amount may be adjusted so that the combined contributions from the maltodextrin and the binder, provide the carbohydrate amount for the formula or dosage amount of the formulated components of the food product.

According to some embodiments, the binder may comprise animal or non-animal based components, and may for example, comprise fats, carbohydrates, proteins (e.g., albumin), and the like.

According to some embodiments, the binding agent may be a gel or sol. According to some embodiments, the sol may be formed of a polymeric carbohydrate gel precursor that is capable of producing a continuous thermostable gel containing a modifying agent. According to some embodiments, the technique of forming a polymeric, carbohydrate gel may be carried out in accordance with any suitable method, including, for example, the methods disclosed in Rivoche, U.S. Pat. Nos. 2,786,764 and 2,791,508; Green et al., U.S. Pat. No. 2,992,925 and Ishler, U.S. Pat. No. 3,093,483, which teach techniques in formation of a thermostable gel, including a polymeric carbohydrate gel, the disclosures of which are herein incorporated by reference. The production of an amino acid containing food product may be produced using a carbohydrate gel. The amino acid containing products may be formed using the gel production methods contained in one or more of the aforementioned patents.

Some examples of the utilization of a sol as a binder or binder component, include the use of a fortified sol. For example, the food product replicas or compositions may be produced by fortifying a sol, and preferably fortifying the sol during its production with the nutritional compositions, or one or more components of the nutritional compositions. Food replicated products and other desirable products can be produced by forming fibers from the sol of the polymeric carbohydrate gel precursor. The carbohydrate gel precursor may produce a continuous thermostable gel containing a modifying agent. Products may be formed having unique textures and eating qualities, which may be accomplished by aggregating fibers to form fibrous compositions. Modifying agents also may be added to the carbohydrate composition to form the sol. Examples of modifying agents may include various modifiable or inert proteinaceous materials, various farinaceous materials, as well as other non-proteinaceous materials such as carbohydrates, fats, colors, flavors, seasonings and plasticizers. The nutritional components also may be added to provide modifications to the gel. These nutritional components of the nutritional compositions may comprise, alone or with other components, modifying agents are capable of actually modifying the gel forming characteristics of the aforesaid gel precursors. Some of the compositions that may be added to form the sol or gel, may include additives which carried by such gel precursors, such as for example compositions comprising raw farinaceous doughs like raw wheat flour dough, raw corn flour dough, raw starch doughs, and the like, calcium caseinate, plasticizers, fats, and the like. The gel or sol may also include additives such as relatively inert filler materials, colors and flavors, inert proteins, such as various seed meals, cottonseed meal, soy bean meal, and farinaceous materials such as cooked wheat flour, corn flour, starches, and the like. The compositions preferably include the amino acids in the desired ratio to provide protein for the food composition or meat substitutes of the invention. The gel or sol represents one way of delivering the amino acid compositions into a food product or food replica or food facsimile product, such as a meat substitute. The binding agents may include modifying agents that either modify the gel forming characteristics of the gel precursors or that may modify the eventual eating quality of the product to be produced using the gel or sol, and including use of the gel or sol as a precursor to a final food product, food replica or facsimile.

The fiber formation may be carried out using the nutritional components, such as the amino acids, and other components. For example, after the fibers are formed, they may be treated to gel the precursor and transform the farinaceous and/or proteinaceous material, if present and of a modifiable nature, from its sol state into a substantially solid state. The process may carry out gelation of the polymeric carbohydrate gel precursor to either precede or take place concurrently with the modification of the farinaceous and/or proteinaceous materials dispersed throughout the sol when such materials are present.

According to some embodiments, when the proteinaceous materials are dispersed in the sol, the nature of the composite fiber resulting upon gelation of the precursor and either coagulation or precipitation of the proteinaceous material is rubbery and elastic and considerably more meat-like in character than gel fibers formed from the polymeric carbohydrate gel precursors alone.

According to some preferred embodiments, the amino acids are added to the sol and are dispersed therein. In addition, the other components of the formulation, such as Vitamins, minerals, and botanical may be added or included in the sol. Aggregation of the amino acids (and in some embodiments one or more other components), are added to the sol. Aggregation of the fibers, aligns the amino acid proteins.

For example, the product, including the gel product may be fiber oriented by alignment of the polymer chains along the longitudinal axis of the fiber or alignment of the polymer chains in an essentially parallel relationship to one another. This aligning or orienting is preferably implemented to reduce the intermolecular spacing between the polymer chains, and decrease the cross-sectional fiber diameter, and increase the tensile strength and/or firmness of the fiber. Some examples of fiber orienting techniques that may be employed include the use of collecting reels that pick up the fibers from a gelling bath at a rate faster than the liquid stream is extruded (e.g., from a source, such as a spinning orifice) so as to cause an elongation or stretching of the fibers in order to orient the polymer chains. The fibers or composition may be stretched with any suitable apparatus, including wheels, which for example, may comprise one or more stretching wheels positioned in line following a collecting reel. For example, some stretching reels are configured to rotate at progressively increasing surface speeds in order to further stretch or elongate the fibers. Alternatively, the fibers may be stretched during the extrusion process, such as for example, by forcing the sol through a spinnerette followed by setting the extruded sol in a coagulating bath (as may be known in the art). The texture, such as for example, chewiness or rigidity, or softness, may be manipulated or regulated to provide a food product that has desired textures, hardness or softness, or stretchability or compaction. The present food and beverage products, including the meat replicas, may be produced using the gellable composition, including advantageously, the orientation of the polymeric carbohydrate chain that imparts a chewy characteristic to the fibers which becomes more evident as the amount of orientation to which the fiber has been subjected is increased. This chewy characteristic contributing to the over-all desired textural characteristics of the product is very desirable.

Food compositions also may be made from the gel precursor, and the gel precursor also may be formulated to include components that affect or regulate the eating qualities such as chewiness (which may be obtained when proteinaceous materials like soy bean meal, peanut meal, fish protein meal, and agglomerates thereof are incorporated in the gel precursor sol and are substantially uncoagulable or unprecipitable). Another property may be provided by the use of farinaceous materials, which when dispersed in the sol, the nature of the composite fiber resulting upon gelation of the precursor and cooking of the farinaceous material may be crisp and friable. The aforementioned are examples of providing a gel composition as a vehicle for providing the nutritional compositions as a food product. The nutritional compositions may be incorporated in whole or part, with some of the components of the composition being introduced into and provided as part of the gel, and other components provided upon the further use of the gel with one or more other components to form the food product.

According to other embodiments, the compositions may include a vegetable juice, a vegetable puree, a vegetable extract, a fruit juice, a fruit puree, or a fruit extract. The vegetable and/or fruit extracts may be those provided with the formulations as a flavoring and/or masking agent. In addition, or alternatively, the vegetable juice, vegetable puree, vegetable extract, a fruit juice, a fruit puree, or a fruit extract can be a Cucumis juice, puree, or extract (e.g., a juice, puree, or extract from a cucumber or a melon). The vegetable juice, vegetable puree, vegetable extract, fruit juice, fruit puree, or fruit extract may be cooked or otherwise treated to denature proteins before adding to the food or beverage compositions. According to some other embodiments, the flavoring agent for a food or beverage composition may include or optionally or additionally may further contain a flavoring agent in the form of a yeast extract, a protein hydrolysate, or an oil. These and/or other flavor compounds or flavor precursors may be added to one or more of the components of the food or beverage product, such as for example, the amino acid mixture (to provide a flavored amino acids mixture), the binder, or other component, or may be added together with one or more other components of the composition.

The nutritional food and beverage compositions may include fats, as discussed. Examples of non-animal fats include plant derived oils, algal oils, or oils from bacteria or fungi; and plant derived oils including coconut oil, mango oil, sunflower oil, cottonseed oil, safflower oil, rice bran oil, cocoa butter, palm kernel oil, palm fruit oil, palm oil, soybean oil, rapeseed oil, canola oil, corn oil, sesame oil, walnut oil, almond oil, flaxseed, jojoba oil, castor, grapeseed oil, peanut oil, olive oil, borage oil, algal oil, avocado oil, hemp seed oil, fungal oil, black currant oil, babassu oil, shea butter, mango butter, wheat germ oil, blackcurrant oil, sea-buckhorn oil, macadamia oil, saw palmetto oil, conjugated linoleic oil, arachidonic acid enriched oil, docosahexaenoic acid (DHA) enriched oil, eicosapentaenoic acid (EPA) enriched oil, or margarine. Oils may include hydrogenated oils (e.g., a hydrogenated vegetable oil) or non-hydrogenated oils. The fats also may include oil fractions which may be used in combination with an oil. For example, an oil fraction, such as stearin (e.g., palm stearin) may be used in combination with coconut oil.

According to preferred embodiments, the preferred embodiments of the present compositions are produced from materials that have no residues of hormones, antibiotics, pesticides, glyphosate or other toxins (as are otherwise found in many plant and animal protein concentrates). The present compositions provide a substitute to dietary protein, which is provided to supplement or replace an individual's other sources of protein. The present compositions are designed to benefit physiological, as well as mental acuity. According to preferred implementations, administering the compositions, such as for example, by ingesting them on a regular basis for a period of several months, or even much sooner, is the target for establishing benefits (though some benefits may be more immediate if a deficiency in one or more ingredients exists). The compositions may be provided in an ingestible form (which may be powdered, tableted, liquid, or concentrate), which may be taken one or more times daily, and over a course of several weeks, months or years.

The present compositions are formulated with a proprietary formulation of amino acids. The formulations include essential amino acids (EAA) and nonessential amino acids (non-EAA), which are provided in a balance with specific nutritive compositions. Preferred embodiments also provide ratios of the EAA and non-EAA. The compositions according to the invention are based on amino acids, and providing ratios and percentages of these amino acids, including EAA and non-EAA together with other substances, such as vitamins or components known to supply such vitamins. The present formulations may include the aforementioned compounds as active ingredients, and the present compounds also may include one or more inactive ingredients, such as for example excipients and/or diluents for administration to an individual, (e.g., in the form of a beverage or drink, or in a pill or capsule form). For example, some preferred excipients include starchy carbohydrates and low molecular weight non-starchy carbohydrates. Other inactive ingredients may include one or more of flavorings, stabilizing agents, and binders. For example, formulations may include citric acid, stevia, xanthan, natural flavors (fruit concentrates or juices), e.g., Luo han guo, flavorings, and the like. Masking agents for preserving flavors and eliminating off flavors and odors over time, may be included as part of the inactive ingredients. For example, natural masking flavor agents that are commercially available may be used (e.g., from Firmenich, Gold Coast and OSF).

The compositions preferably may include one or more vitamins and substances that assist, for example, in one-carbon metabolism. Some examples of the assisting substances include folate (Vitamin B9), vitamin B12, vitamin B6, riboflavin, methionine, choline and betaine, which are involved in DNA methylation by regulating levels of the universal methyl donor S-adenosylmethionine and methyltransferase inhibitor S-adenosylhomocysteine. The present compositions also may include one or more other nutrients and bioactive food components, and in particular substances that may modulate epigenetic patterns by altering the levels of S-adenosylmethionine and S-adenosylhomocysteine or directing the enzymes that catalyse DNA methylation and histone modifications. Some examples of these substances include retinoic acid, resveratrol, curcumin, sulforaphane and tea polyphenols.

Vitamin B3, which, according to preferred embodiments, is present in the compositions of the invention, acts as a co-factor in both steps, where dietary folate is converted to dihydrofolate (DHF) via the dihydrofolate synthase (DHFS) enzyme, and where the dihydrofolate (DHF) is then converted to tetrahydrofolate (THF) by the dihydrofolate reductase (DHFR) enzyme.

According to preferred embodiments, the compositions of the invention include as a major ingredient a complex carbohydrate, such as for example, maltodextrin, which according to some preferred formulations is provided as Organic Tapicoa Maltodextrin DE 10. According to composition formulations where a complex carbohydrate is included as part of the formulation, as a major ingredient, the complex carbohydrate, e.g., maltodextrin, represents the largest percentage ingredient of the active ingredients (e.g., by weight percentage of the active ingredients). According to some preferred formulations, the maltodextrin preferably is provided in an amount that is less in weight percent of the total active ingredients, than the weight percent of the combined total amino acids in the formulation. According to preferred embodiments, the maltodextrin preferably may comprise 20 to 35 percent of the weight percent of active ingredients in the formulation.

According to preferred embodiments, compositions according to the present formulation, include proteinogenic amino acids. Preferred formulations include amounts of branched-chain amino acids (BCAAs), the BCAAs comprising isoleucine, leucine, and valine. Preferred compositions also include, in addition to the BCAAs, additional essential amino acids, including histidine, lysine, methionine, phenylalanine, threonine, and tryptophan, which preferably are present in the composition and comprise further proteinogenic amino acids in addition to the BCAAs.

Preferred compositions also include non-essential amino acids. In particular, the non-essential amino acids of the preferred embodiments of the present formulations include arginine, cysteine, cystine, glycine, proline, and tyrosine.

The BCAAs typically are considered to be responsible for improving muscle growth and reducing fatigue. The present formulations provide a composite of ingredients that seeks to maximize or enhance the body's capability to use the active ingredients, such as, for example, the amino acids, including the BCAAs. According to preferred embodiments, the formulation includes BCAAs. According to preferred embodiments, the BCAAs may comprise up to about 25%, and preferably about 15-22% by weight of the active ingredients, where the BCAAs component of the formulation are present in a ratio (amongst themselves) by weight percent of about 50% leucine, and about 25% each of isoleucine and valine. According to preferred compositions, the formulation includes leucine provided as the most abundant amino acid component by weight of the percentage of the amino acids in the formula, and one of the two most abundant active ingredients of the formulas (the other being a maltodextrin according to some preferred embodiments). For example, preferred embodiments of the composition include leucine present in an amount of about 7 to 12% weight percent of the active ingredients, and preferably between 8 and 10%. The compositions include isoleucine and valine, which preferably are present among the active ingredients in weight percentages of about 3.5 to 5.5%. According to preferred embodiments, the isoleucine and valine are provided in equal amounts. The composition preferably may provide valine and isoleucine components in an amount each that is about 40-60% of the weight percent of the amount of leucine. The amount of lysine in the compositions of the invention may be similar to the amounts of isoleucine and valine. The amino acids isoleucine, valine and lysine are among the second most abundant amino acids provided in the formula (with leucine being the most abundant amino acid component). The majority amino acids in the formulations are leucine, isoleucine, lysine and valine.

The preferred compositions also include threonine, proline, glycine, and arginine, which preferably are provided in a weight percent of active ingredients, of about 2 to 7%.

According to preferred embodiments, the composition also includes additional amino acids. The additional amino acids present in preferred formulations include cysteine, histidine, phenylalanine, methionine, cystine, tyrosine, tryptophan, threonine and theanine. These amino acids may be present in amounts up to about 7% by weight of the active ingredients (when the active ingredients include medium chain triglycerides, maltodextrin and lecithin, as in Formulation Example 3). Additional embodiments are provided herein wherein these amino acids are provided in ranges, ratios or different amounts.

Preferred compositions also include a cell protecting component, and a neuro-regenerative component. According to preferred embodiments the cell-protecting component comprises phosphatidylserine (a phospholipid), and the neuro-regenerative component comprises Vitamin B3 (niacinamide). These components are provided with the preferred amino acid formulation. The Vitamin B3, as referenced herein, increases the body's ability to promote healthy nerve cells, and in the present composition, with the amino acids, facilitates and/or improves neuron health, as well as regeneration and repair of neurons. The Vitamin B3 may boost nerve cell energy metabolism. The present compositions provide the proteins in the form of amino acids, which can supply energy for use in cell metabolism. The vitamin B3 boosts the nerve cell energy metabolism, so that a synergistic effect is realized where proteins that supply cellular energy from the amino acid blend are utilized by the nerve cells, and promote repair and/or regeneration of the nerve cells, or components of the nervous system, as well as the brain. Vitamin B3 also may promote synthesis of tetrahydrofolate (THF). The cell protecting component, phosphatidylserine, preferably is provided in the form of Phosphatidylserine 70% as SerinAid® (Chemi Nutra).

The compositions also include minerals and botanicals, which contribute to metabolic operations within the body that promote the utilization of the proteins.

In addition to the amino acids comprising the formula, according to preferred embodiments, additional components are included, any may include one or more or combinations of vitamins, minerals and/or botanicals, such as the following, which may be provided as active ingredients: Curcuma longa extracts as Turmacin®, Curcumin C3 Complex® (NLT 64:1 standardization ratio), Curcumin C3 Reduct (Tetrahydrocurcumin standardized for 95% total curcuminoids), turmeric (95% curcuminoids), curcurmin, TheraCurmin®, LongVida®, silymarin, trans-resveratrol, Taurine, Alpha-Glyceryl Phosphoryl Choline (as choline-alFLOWsecrate®, AlphaSize® or other forms of alpha GPC), PhosphatidylSerine (preferably SerinAid®), Vitamin K2 MK7 (preferably MenaQ7® MK7 form of vitamin K2), Vitamin C (ascorbic acid), thiamine (Vitamin B1), Vitamin B3 (Niacinamide) Vitamin B12 (preferably methylcobalamin), Vitamin B6, Vitamin B9 (preferably Methylfolate), selenomethionine SeleniumSeLECT®, a form of boron, such as boric acid and/or boron citrate, boron glycinate, and/or boron aspartate, a magnesium source (such as magnesium citrate), ginko biloba, complex carbohydrate (e.g., maltodextrin), medium chain triglycerides, (e.g., MCT Neobee® 50-70% powder), a fatty substance, such as lecithin (e.g., sunflower or canola lecithin). Preferably, the active ingredients are obtained from natural organically farmed or wildcrafted sources, to facilitate bio-availability and purity of the substances and for use in association with other components of the formulation or in the individual's body to be readily available as needed. According to preferred embodiments, non-amino acid active ingredients are present in amounts of less than about 14% of the active ingredients (by weight), with the exception of the complex carbohydrate (e.g., maltodextrin), magnesium citrate, and an energy source, namely, an energy source that serves as an alternative energy source to glucose, such as medium chain triglycerides (e.g., MCT Neobee® powder 50-70%, commercially available from Chempoint®, Bellevue, Wash.), which are each provided in amounts greater than 1%. According to preferred embodiments, with the maltodextrin, MCT, and lecithin included in the composition as active ingredients, the compositions include up to about 8%, and preferably about 4 to 10% by weight of magnesium citrate, and up to about 12%, and preferably about 6-11% of an energy source, such as the MCT Neobee® powder 50-70%, and up to about 35%, and preferably about 20-33% maltodextrin.

The composition preferably includes a source of methyl groups, which is needed for many steps in metabolism, including for example to synthesize phosphatidylcholine and sphingomyelin, two major phospholipids vital for cell membranes. According to preferred embodiments a methyl group source comprises choline. Preferred embodiments provide choline on the form of AlphaSize® 50WSP Alpha-Glyceryl Phosphoryl Choline (ALPHA GPC). Choline preferably is present as an active ingredient up to about 2% and preferably, about 0.8 to 2% by weight of the active ingredients of amino acids, vitamins, minerals and botanicals.

In addition to the active ingredients, the composition preferably includes other ingredients (inactive ingredients), which may be used to preserve, flavor, mask flavors and maintain the consistency of the compositions. The inactive ingredients may be added as desired for a suitable flavor, or as an excipient or for tableting, or for providing a drinkable formulation. According to some preferred embodiments, the inactive ingredients comprise about 18 to 22% of the composition (by weight), with the active ingredients comprising the balance (e.g., 78 to 82%), see e.g., Formulation Example 3, where maltodextrin, MCT, and lecithin are included among the active ingredients), and where inactive ingredients comprise Natural Masking Flavor, natural citrus flavoring, citric acid, stevia 97-99%, luohan guo 50%, Magrosides by UV, Faststir Xanthan EC.

According to some embodiments, the compositions include a complex carbohydrate, such as, maltodextrin, as a major component, and according to preferred embodiments, maltodextrin is present in an amount of the active ingredients, by weight percent, of about 20 to 33% of the active ingredients. According to preferred embodiments, the ratio of maltodextrin to the branched chain amino acids of the formula, is about 3:2 (maltodextrin to BCAAs), meaning that, according to a preferred composition, the maltodextrin may comprise about 20 to 33% (by weight) of the active ingredients, while the BCAAs comprise about 13 to 22% (by weight) of the active ingredients.

According to some embodiments, compositions include EAAs, non-EAAs, and other non-amino acid components. Exemplary compositions may include about 32 to 34% essential amino acids as a weight percent of the active ingredients (when including the MCT, lecithin, and maltodextrin as active ingredients), and about 9 to 11% of non-essential amino acids as a weight percent of the active ingredients (when including the MCT, lecithin, and maltodextrin as active ingredients, as in Formulation Example 3). A preferred ratio of EAAs to non-EAAs present in the active ingredients, by weight, is about 3:1 to 4:1, with a most preferred ratio being about 3:1 weight percent of EAAs to non-EAAs.

According to a preferred embodiment, the formulation may be provided to include as active ingredients, EAAs of: isoleucine, leucine, valine, methionine, phenylalanine, threonine, tryptophan, histidine, and lysine.

Preferred compositions also include non-essential amino acids. In particular, the non-essential amino acids of the preferred embodiments of the present formulations, include arginine, cysteine, cystine, glycine, proline, and tyrosine.

According to preferred embodiments, of the amino acids in the formula, the essential amino acids are present up to about 77% by weight of the amino acid content, and preferably in an amount of about 75% by weight, and the non-essential amino acids up to about 25%, and preferably about 23% by weight of the amino acid content. The essential amino acids may comprise about 33% by weight of the active ingredients of the formula, such as the Formulation Example 2, where the active ingredients include amino acids, Vitamins, minerals and botanicals, but without the complex carbohydrate (maltodextrin), triglycerides (MCT), fat (lipids), (and preferably about 27%, when the formula active ingredients are amino acids, vitamins, minerals and botanicals, and complex carbohydrate (maltodextrin), triglycerides (MCT), fat (lipids)). The non-EAAs may comprise about 8 to 10% by weight of the active ingredients of the formula (when the formula active ingredients are amino acids, vitamins, minerals and botanicals, and complex carbohydrate (maltodextrin), triglycerides (MCT), fat (lipids)). According to some embodiments, the essential amino acids may be provided in a ratio, where amongst themselves, leucine is about 33% of the EAA in the formula, lysine, isoleucine and valine are about 16 to 17% each (or, alternatively, in combination about 48% to 51% of the EAAs), threonine about 9%, with histidine, methionine and phenylalanine, about 2.5 to 3% each (with some preferred embodiments histidine being above 3% and each of methionine and phenylalanine being below 3%), and with tryptophan being less than 1% (and in some embodiments about 0.5%). Also present in the formulation are non-EAAs. For example, non-EAAs, arginine, cysteine, cystine, glycine, proline, and tyrosine, according to preferred embodiments, may be present in ratios among themselves, with arginine and glycine, being about 25% to 26% of the non-EAAs (for each of them), and with proline being about 29-31% of the non-EAAs, cysteine being about 9-11% of the non-EAAs, and cysteine being about 5 to 7% of the non-EAAs, and tyrosine being about 2 to 3% of the non-EAAs.

According to a preferred embodiment, the EAAs may comprise about 74 to 77% of the total amino acids of the formula, while non-EAAs may comprise about 23 to 26% of the total amino acids in the formula. The formula may further include amino acid derivatives, which are in addition to the total amino acids in the formula. The food products preferably also are formulated to be produced having the aforementioned ratios of EAAs to non-EAAs. For example, according to some preferred embodiments, food products of the invention may be products wherein the EAAs are provided to comprise about 74% to 77% by weight of the total amino acids of the food product, and wherein the non-EAAs comprise about 23% to 26% by weight of the total amino acids of the food product.

According to some alternate embodiments, the food product is provided with at least the composition of amino acids set forth in Tables 1 and 2, so that a minimum of the amino acids are present.

According to some preferred embodiments, the composition may include the following EAAs, which are listed in Table 1:

TABLE 1

L-Leucine
L- Histidine
L-Isoleucine
L-Lysine
L-Methionine
L-Phenylalanine
L-Threonine
L-Tryptophan
L-Valine

According to embodiments, the composition also contain non-EAAs:

TABLE 2

L-arginine
L-cysteine
L-cystine
L-glycine
L-proline
L-tyrosine

According to some further embodiments, the formulation includes vitamins. According to a preferred embodiment, a formulation comprising the EAAs and non-EAAs further includes vitamins. For example, a formulation is produced having vitamins including Vitamin C (ascorbic acid), Vitamin K2 MK7 (MenaQ7®-menaquinone-7), thiamine (Vitamin B1), Vitamin B3 (Niacinamide), Vitamin B12 (methylcobalamin), Vitamin B6, and Vitamin B9 (Methylfolate). The vitamin amounts in the formula preferably are less than 1% by weight of the active ingredients and may be much less, each being present on the order of tenths to thousandths of a percent of the active ingredients, with the exceptions being Vitamin K2 MK7 about 0.2 to 1.5%, and Vitamin C being about 0.5%, A formula is produced, according to a preferred embodiment, and includes, as active ingredients, the EAAs of Table 1 the non-EAAs of Table 2, and the following vitamins in Table 3.

TABLE 3

Vitamin C
Vitamin K2 MK7
Vitamin B1
Vitamin B3
(Niacinamide)
Vitamin B12
Vitamin B6
Vitamin B9

According to a preferred embodiment, the above formula of amino acids and vitamins also includes phosphatidylserine (in the form of PhosphatidylSerine 70% as SerinAid®), preferably provided in an amount of 0.50% by weight of active ingredients, or about 1% by weight relative to the weight of amino acids in the composition, and boron (in the form of boric acid), in an amount of 0.20% by weight of active ingredients or about 0.4% by weight relative to the weight of amino acids in the composition.

As referenced, the formula may be ingested along with one or more additional substances, such as, for example, one or more complex carbohydrates, and/or fats, and preferably both. However, according to some preferred embodiments, the compositions are formulated to include the complex carbohydrate and/or fat as part of the formula.

According to a preferred embodiment, the above formula preferably includes a major component of a complex carbohydrate, which, according to some embodiments, may make up 25% of the formula. According to a formulation, the complex carbohydrate comprises maltodextrin, which is added to the above formulas.

The above formulas also may include a fatty substance, such as for example, lecithin, and, according to some preferred embodiments, lecithin may be provided in amounts from about 0.5 to 2% of the active ingredients by weight, with a preferred amount being about 1%.

According to a preferred embodiment, one or more additional substances are provided as active ingredients in the formula, which may comprise one or more substances such as minerals, botanicals and/or triglycerides (preferably medium chain triglycerides—MCT).

According to some preferred embodiments, additional active ingredients may include the following substances, which may be included as part of the active ingredients of the formula: turmeric (Turmacin) 0.4%, turmeric (95% curcuminoids) 1.15%, silymarin 0.8%, resveratrol (trans-resveratrol) 0.8%, taurine 2.3%, choline (AlphaSize® (alpha GPC)) 0.58%, Curcuma longa extracts (Theracumin) 0.4%, (Turmacin) 0.4% Curcumin C3 Complex (NLT 64:1 standardization ratio) 1.15%, Curcumin C3 Reduct (Tetrahydrocurcumin standardized for 95% total curcuminoids) 0.4%, selenomethionine 0.0015%, ginko biloba 0.15%, MCT (Neobee® 50-70% powder) 10%, lecithin (e.g., sunflower lecithin) 1%. A formula was prepared containing these aforementioned additional active ingredient substances in the listed weight percentages, along with the EAAs listed in Table 1, the non-EAAs listed in Table 2, according to the relative ratios of amino acids (EAA to non-EAA, and particular amino acid relative ratios), and the vitamins of Table 3. According to preferred embodiments the vitamins were less than 1% by weight of active ingredients, and for vitamins other than Vitamin C and Vitamin K2 MK7, were added in amounts of 0.1 to 0.0001%.

In addition the formulas preferably include an amount of magnesium citrate. Preferred embodiments include magnesium citrate in an amount of about 5 to 8% of the active ingredients.

An exemplary formula, according to a preferred embodiment of a formula that includes the EAAs and non-EAAs from Tables 1 and 2 provided in accordance with the ratios discussed above, the vitamins of Table 3, and further includes magnesium citrate in an amount of about 6.8% by weight of active ingredients. The formula preferably also includes the substances: turmeric (95% curcuminoids) 0.5%, silymarin 1%, resveratrol (trans-resveratrol) 1%, taurine 2.3%, choline (choline-AlphaSize (alpha GPC)) 0.72%, Curcuma longa extracts, Theracumin (NLT 33% curcuminoids) 0.5%, turmeric (Turmacin) 0.5%, Curcumin C3 Complex (NLT 64:1 standardization ratio) 1%, Curcumin C3 Reduct (Tetrahydrocurcumin standardized for 95% total curcuminoids) 0.4%, selenomethionine (preferably as SeleniumSeLECT 39% selenium) 0.002%, ginko biloba 0.2%, free-flowing, micro-encapsulated medium chain triglycerides oil, preferably provided as MCT Neobee® 50%-70% powder, 9.6% (Stepan Company), and lecithin (e.g., sunflower lecithin) 1%.

A component of the formulas according to preferred embodiments is a complex carbohydrate, such as maltodextrin. According to an exemplary formula, a large portion of the formula balance is the complex carbohydrate, and preferably may be maltodextrin, which was provided in an amount of about 32% of the active ingredients (for the formula comprising the components of Tables 1, 2 and 3, in which maltodextrin is present as an active ingredient). According to preferred embodiments, there is preferably a 1:1 ratio of maltodextrin to the essential amino acids in the formulation, and wherein maltodextrin is present at about 75% of the amount of total amino acids. According to preferred embodiments, total amino acids are present in the formulations in an amount of about 40 to 46% of the active ingredients (see e.g., Formulation Example 3). A composition dosage of 13 g provides amino acids of about 5 g.

According to a preferred embodiment, the supplement formula includes the substances listed in Tables 1, 2 and 3, and phosphatidylserine (in the form of PhosphatidylSerine 70% as SerinAid®), boric acid, magnesium citrate, as well as the additional active ingredients, and maltodextrin.

According to some preferred embodiments, a formula comprises the substances and components including amino acids, of Tables 1, 2 and 3, in the relative ratios indicated, and phosphatidylserine (in the form of PhosphatidylSerine 70% as SerinAid®), boric acid, and vitamins in the amount of less than 1% each, the vitamins including: Vitamin C (ascorbic acid), Vitamin K2 MK7, thiamine (Vitamin B1), Vitamin B3 (Niacinamide), Vitamin B12 (methylcobalamin), Vitamin B6, and Vitamin B9 (Methylfolate); and 5 to 8% magnesium citrate and about 25 to 40% maltodextrin. According to a further embodiment, the previously discussed formula was prepared, and further included the following substances: turmeric (turmacin) 0.5%, turmeric (95% curcuminoids) 0.5%, silymarin 1%, resveratrol (trans-resveratrol) 1%, taurine 2.3%, choline (AlphaSize® (alpha GPC)) 0.72%, Curcuma longa extracts (Theracumin-NLT 33% curcuminoids), (theracumin) 0.5%, curcumin c3 complex (NLT 64:1 standardization ratio) 1%, Curcumin C3 Reduct (Tetrahydrocurcumin standardized for 95% total curcuminoids) 0.5%, selenomethionine 0.002%, ginko biloba 0.2%, MCT Neobee® 50%-70% powder 9.6%, lecithin (e.g., sunflower lecithin) 1%.

The formulas described herein preferably include inactive ingredients, among which may include flavorings, texture enhancers, storage enhancers, and other components that may aid in forming the tablet, liquid or other form of the composition so it may be administered to an individual. According to some embodiments, the inactive ingredients may comprise 15 to 25% of the weight total of formulation, with the active ingredients comprising 75 to 85% of the total ingredients by weight.

According to some further embodiments, the formulas may be prepared without the complex carbohydrate, namely, the maltodextrin. In these preparations, the weight percent of the active ingredients may remain the same, so the components present in the formula will have the relative weight percents (e.g., adjusted to reflect the absence of the maltodextrin). According to these alternate embodiments, the supplement may be provided for use where a person will also be expecting to ingest food, such as a food that comprises a complex carbohydrate. In addition, the compositions also may be prepared without fats or lipids. For example, although some formulations include lecithin (e.g., sun flower lecithin) and/or triglycerides (MCT, e.g., Neobee®), the weight percent of the active ingredients may remain the same, so the components present in the formula will have the relative weight percents (for active ingredients, wherein the weight percents may be adjusted for the total formula to reflect the absence of the lecithin and/or MCT). According to these formulations, where no fats or lipids are present in the formula, the supplement may be taken where a person is expecting also to consume fats or lipids with the composition. The formulations therefore may be prepared without maltodextrin (complex carbohydrate) and fats. In the examples where the formula does not include one or more of the carbohydrate or fat, there may be a window within which the complex carbohydrate, and/or fats and lipids may be consumed relative to the time of consumption (e.g., ingestion) of the dietary supplement composition (e.g., amino acid blend, Vitamins, minerals and botanicals).

### EXAMPLES

The following amino acids are provided in the food or beverage composition. The food or beverage composition may include a nutritional fraction that is comprised of the following amino acids as a percent of the active ingredients in the nutritional fraction of the food or beverage composition. The amino acids preferably may be present as a percent of the active ingredients in the food or beverage composition (or as a percent of the nutritional fraction), to constitute about 70 to 85% of the formula, and more preferably about 78% of the formula. The amino acids of preferred compositions preferably comprise the following amino acid blend, in Table A:

TABLE A

L-LEUCINE
L-LYSINE
L-ISOLEUCINE
L-VALINE
L-THREONINE
L-PROLINE
L-GLYCINE
L-ARGININE
L-CYSTINE
L-HISTIDINE
L-PHENYLALANINE
L-METHIONINE
L-CYSTEINE
L-TYROSINE
L-TRYPTOPHAN
L-THEANINE

In the preferred compositions, the amino acids are present in ranges. According to a preferred embodiment, the composition has an amino acid blend, with the blend including the amino acids listed in Table A. The amino acid blend has a preferred composition, with ranges of the amino acid components as follows: with tyrosine, tryptophan and theanine constituting less than 1% each by weight percent of the amino acid blend of the composition; with cystine, histidine, phenylalanine, methionine, and cysteine, comprising from about 1.25 to about 3.5% each by weight percent of the amino acid blend, and with threonine, proline, glycine and arginine, comprising from about 5 to 7% each by weight percent of the amino acid blend; with isoleucine lysine and valine comprising from about 10 to 13% each by weight percent of the amino acid blend, and with leucine comprising the major amino acid of the blend, at about 20 to 27%. According to some embodiments, compositions may be formulated with the amino acid blend conforming to BCAA ratios described herein.

### Formulation Example 1

The following formula is prepared with active ingredients comprising EAAs and non-EAAs, and vitamins, which are indicated as active ingredients and as weight percentages of the active ingredients.
L-LEUCINE
L-LYSINE
L-ISOLEUCINE
L-VALINE
L-THREONINE
L-PROLINE
L-GLYCINE
L-ARGININE
L-CYSTINE
L-HISTIDINE
L-PHENYLALANINE
L-METHIONINE
L-CYSTEINE
L-TYROSINE
L-TRYPTOPHAN
L-THEANINE
THIAMINE (VITAMIN B1)
VITAMIN B3 (Niacinamide)
VITAMIN B6
VITAMIN B9 (Methylfolate)
VITAMIN B12 (METHYLCOBALAMIN)
VITAMIN C (ASCORBIC ACID)
Vitamin K2 MK7 as MenaQ7 Pure 2,000

The above formulation includes the following Vitamins, which are in addition to the amino acid blend of the composition, of Table A. The following Vitamins are represented in Formulation Example 1, above, and include: Thiamine (Vitamin B1), Vitamin B3 (niacinamide), Vitamin B6, Vitamin B9 (Methylfolate), Vitamin B12 (Methylcobalamin), Vitamin C (Ascorbic Acid), Vitamin K2 MK7 (as MenaQ7 Pure 2,000). According to a preferred formulation, the composition provides the amino acid blend in an amount of about 5 g, and the vitamin portion containing the vitamins listed, is about 0.1 to about 0.2 g, providing a composition of about 5.1 to 5.2 g of amino acids and vitamins. Preferably, the composition includes Vitamin B3 (niacinamide) in an amount of about 0.075 to 0.4% by weight of active ingredients, and about 0.2 to about 0.6 mg of the amino acid Vitamin mixture (of the 5.1 to 5.2 g).

### Formulation Example 2

According to another embodiment, the above formulation, with the amino acid blend and vitamins, is further enhanced with minerals and botanicals. The composition in Formulation Example 2 is indicated below, and includes the components set forth in Formulation Example 1 above, and further includes additional nutrients, including minerals and botanicals.
L-LEUCINE
L-LYSINE
L-ISOLEUCINE
L-VALINE
L-THREONINE
L-PROLINE
L-GLYCINE
L-ARGININE
L-CYSTINE
L-HISTIDINE
L-PHENYLALANINE
L-METHIONINE
L-CYSTEINE
L-TYROSINE
L-TRYPTOPHAN
L-THEANINE
L-CARNITINE
THIAMINE (VITAMIN B1)
VITAMIN B3 (Niacinamide)
VITAMIN B6
VITAMIN B9 (Methylfolate)
VITAMIN B12 (METHYLCOBALAMIN)
VITAMIN C (ASCORBIC ACID)
Vitamin K2 MK7 as MenaQ7 Pure 2,000
PhosphatidylSerine 70% as SerinAid
AlphaSize® 50WSP Alpha-Glyceryl Phosphoryl Choline (ALPHA GPC)
TURMERIC (TURMACIN)
CURCUMA LONGA EXTRACT-Theracurmin (NLT 33% Curcuminoids)
CURCUMIN C3 COMPLEX (NLT 64:1 Standardization Ratio of Curcuminoids)
CURCUMIN C3 REDUCT (Tetrahydrocurcumin standardized for
95% total curcuminoids)
GINGKO BILOBA 24%
SILYMARIN
TRANS-RESVERATROL
TAURINE
L-SELENOMETHIONINE as SeleniumSeLECT 39% Selenium
Boric Acid
MAGNESIUM CITRATE 16% (Nutrisol Brand)

The additional nutrients, in particular, minerals and botanicals, preferably comprise about a 1.0 to about 1.6 g addition to the composition of Formulation Example 1. According to a preferred composition, as represented by Formulation Example 2, the composition is about 6.1 to 6.8 g.

In Formulation Example 2, the major component of the additional nutrients, minerals and/or botanicals, comprises magnesium citrate 16% by weight (Nutrisol Brand), which is about 0.4 to 0.8 g of the 1.0 to 1.6 g of the additional nutrients, botanicals and minerals, that are provided in Formulation Example 2. The reminder of the nutrients, botanicals and/or minerals, are about 0.1 to about 1.1 g, and preferably include at least an amount of about 30 mg of phosphatidylserine, which preferably is provided in an amount of about 0.4 to 1.2% by weight of the composition, Formulation Example 2. In addition, as part of the remaining 0.1 to about 1.1 g of botanicals and minerals, boron is provided, and in accordance with the Formulation Example 2, is in the form of boric acid, which according to a preferred composition formulation is provided in an amount of about 20 mg of boric acid, and, according to compositions, preferably, boric acid may be provided in an amount of about 0.4 to 1.2% by weight of the composition, Formulation Example 2. According to preferred embodiments, the boron and Vitamin K2 MK7, preferably are provided in the composition in a ratio, wherein the amount of boric acid is present in an amount of about 1 mg for every 2.25 mg Vitamin K2 MK7 (as MenaQ7 Pure 2,000).

As further indicated in Formulation Example 2, the composition includes a turmeric portion, which is represented by three components forming the turmeric component or portion of the composition. According to a preferred embodiment, the turmeric component of the composition, preferably includes the following relative weight percents of turmeric components (relative to each other) based elements: 16.7% Turmacin, 16.7% Curcuma longa Extract-Theracurmin (NLT 33% Curcuminoids), and 50% Curcumin C3 Complex (NLT 64:1 Standardization Ratio of Curcuminoids), 16.7% Curcumin C3 Reduct (Tetrahydrocurcumin standardized for 95% total curcuminoids). The turmeric components in the Formulation Example 2, preferably comprise about 300 mg of the 0.9 to 1.5 g of botanicals and minerals. According to preferred embodiments, the turmeric composition may be from about 4 to 6% of the active ingredients of Formula Example 2.

### Formulation Example 3

The above formulas may be formulated with additional components, which may be active ingredients and/or inactive ingredients. According to some formulations, the maltodextrin, MCT and lecithin may be considered active ingredients. In some formulations, according to some embodiments, in addition to the active ingredients set out above in Formulation Example 1, above, and/or the active ingredients set out in Formulation Example 2, above, additional ingredients, which may include carbohydrate (preferably complex carbohydrate), and triglycerides (MCT), fats (e.g., lecithin), and inactive ingredients such as flavorings, texturing compositions, storage enhancers, may be included in a composition with the ingredients (of Formulation Example 1, and/or Formulation Example 2.

According to preferred compositions, an exemplary composition that includes the active ingredients of Formulation Example 2, above, plus additional ingredients, is represented by Formulation Example, 3. An exemplary composition, represented in Formulation Example 3, below, provides the active ingredients of Formulation Example 2, to constitute about 6 g to 6.7 g, plus the additional components present in Formulation Example 3, which comprise an additional about 7 g to 7.8 g. The additional ingredients comprise, as the major amount of additional ingredients, maltodextrin (Organic Tapicoa Maltodextrin DE 10), which is provided to constitute about 40 to 50% of the additional ingredients, with MCT constituting about 12 to 16% of the additional ingredients, with the remainder of the additional ingredients being made up of flavorings, masking agents, and texturizing compositions.
L-LEUCINE
L-LYSINE
L-ISOLEUCINE
L-VALINE
L-THREONINE
L-PROLINE
L-GLYCINE
L-ARGININE
L-CYSTINE
L-HISTIDINE
L-PHENYLALANINE
L-METHIONINE
L-CYSTEINE
L-TYROSINE
L-TRYPTOPHAN
L-THEANINE
L-CARNITINE
THIAMINE (VITAMIN B1)
VITAMIN B3 (Niacinamide)
VITAMIN B6
VITAMIN B9 (Methylfolate)
VITAMIN B12 (METHYLCOBALAMIN)
VITAMIN C (ASCORBIC ACID)
Vitamin K2 MK7 as MenaQ7 Pure 2,000
PhosphatidylSerine 70% as SerinAid
AlphaSize® 50WSP Alpha-Glyceryl Phosphoryl Choline (ALPHA GPC)
TURMERIC (TURMACIN)
CURCUMA LONGA EXTRACT-Theracurmin (NLT 33% Curcuminoids)
CURCUMIN C3 COMPLEX (NLT 64:1 Standardization Ratio of Curcuminoids)
CURCUMIN C3 REDUCT (Tetrahydrocurcumin standardized for 95% total curcuminoids)
GINGKO BILOBA 24%
SILYMARIN
TRANS-RESVERATROL
TAURINE
L-SELENOMETHIONINE as SeleniumSeLECT 39% Selenium
Boric Acid
MAGNESIUM CITRATE 16% (Nutrisol Brand)
MCT Neobee 70% Powder
Sunflower Lecithin
ORGANIC TAPICOA MALTODEXTRIN DE 10
FIRMENISCH NATURAL MASKING FLAVOR
FIRMENISCH NATURAL MANGO FLAVOR
Citric Acid
Stevia 97-99%
LuoHan Guo 50% Magrosides by UV
Faststir Xanthan EC
Flavoring

According to preferred embodiments, the composition represented by Formulation Example 3 may be provided in a powder form, and stored for daily use. The formulation may have a shelf life of up to at least three years. Preferred compositions are formulated to provide the nutrient components of the formulations presented herein, with a shelf-life of at least 3 years, and greater. The powder preferably is mixed into room temperature water, by placing a dosage amount, such as 13 g of the composition, into the water. The powder is allowed to settle, then mixed (e.g., by stirring) to dissolve it or form a slurry. The admixture may be augmented with juice, or may be blended into a smoothie, or other drink or food. Alternatively, the powder may be admixed with food compositions, provided that the food composition will not subject the powder to excessive temperatures (e.g., heating or baking) that may change amino acid structures.

The invention provides a human dietary supplement comprising a blend of amino acids, each dosage including EAAs and non-EAAs in an amount of about 5 g, with Vitamin K2 MK7 in an amount of about 30 to 60 mg, and with Vitamin B3 (niacinamide) in an amount of about 5 to 30 mg, each dosage taken twice daily, wherein the supplement provides a unit dosage of an effective amount of nutrients to effectively improve muscular and neurological performance, and wherein the dietary supplement is manufactured and supplied to be used over a period of time. Additional compositions are formulated to include the additional Vitamins, as well as botanicals, minerals, as well as MCT, maltodextrin, and lecithin, each as an active ingredient. The compositions may include flavorings, masking agents, and texturizing agents, which do not contribute to the activity of the composition.

The compositions of the invention provide amino acids that the body can metabolize, and do not require detoxification when co-consumed with other protein sources. The present compositions may be co-consumed with other protein sources, and provide the effective benefits, without drawbacks of producing substances or reactions that need to be addressed by further measures or products.

The method of tissue repair, which may include repair of body cells, including cell structures, involves ingesting an amount of the compositions of the invention. As an example, the composition of Formulation Example 3, is consumed in an amount of 13 g per serving, twice daily, where, of the 13 g serving, the amino acids comprise 5 g of the 13 g serving, and the Vitamins comprise about 0.150 g, minerals and botanicals about 0.9 to 1.5 g, and the balance, about 7 g to 7.8 g, including, as the major amount, maltodextrin (Organic Tapicoa Maltodextrin DE 10), which is provided to constitute about 40 to 50% of the balance, with MCT constituting about 12 to 16% of the balance, with the remainder of the balance being made up of flavorings, masking agents, and texturizing compositions. The method involves ingesting the composition, which is taken by mixing the dry powder into water, and taking in that form or with juice (or other blend). The 13 g composition portion is taken twice daily. The method involves taking a daily total therefore of 10 g of an amino acid blend, split into two portions. The amino acid blend includes the blend of EAA and non-EAA, in the composition ratios discussed herein. The amino acid blend also is consumed as part of the formula (e.g., Formulation Example 3) which also contains the vitamins, minerals and botanicals, as well as complex carbohydrate, medium chain triglycerides and fat (lipids). The composition minimizes the digestive energy required for decomposition and absorption of the nutrients by the gastro-intestinal system. Taking the composition, in accordance with the method, increases the body's nitrogen balance to promote the anabolic state in which the body builds or repairs tissue. In addition, the synergistic mixtures of the amino acids, with the other components, provide mechanisms for promoting tissue repair, including cellular repair and regeneration. The method preferably may be used to facilitate restoration of functions, or improve their time for recovery. The present compositions are designed to help reduce existing inflammation in the body, and assist the tissue repair process.

The present compositions provide nutritional building blocks that support brain function, body tone, skin texture, muscle density and increase vitality and strength without the requirement of the use of stimulants.

An important feature of the invention is to provide a method to improve muscle tone, and repair tissue damage or regenerate tissue and other cells, by consuming the dosages of the composition, twice daily 13 g portions, over several weeks or months (which may be continued thereafter). The method involves ingesting the combination of an amino acid blend plus a cell protective compound, which is phosphatidylserine (a phospholipid), and, a neuro-regenerative compound, which is Vitamin B3 (niacinamide). The amino acids provide the proteins that may be useful to build cell structures. However, the cell protective compound and neuro-regenerative compound when consumed together with the amino acid blend and the other minerals and botanicals in the composition, facilitate stimulation of the re-growth and repair of neurons and their connections (dendrites, synapses and axons) to organs, nerve cells and muscles. Taking doses of the present compositions stimulate the production of mitochondria in the cells, and increasing the mitochondrial density in each cell type, but especially in those of the muscle cells. The compositions improve metabolic function, and are designed to facilitate conversion at the mitochondria of nutrients into Adenosine Tri-Phosphate (ATP), which is the fuel used to produce energy in the body.

Preferably the method includes taking the composition with the Vitamins set out in Formulation Example 1, and the minerals and botanicals set out in Formulation Example 2, and according to preferred implementations, taking the composition that includes the amino acids, Vitamins, nutrients including the minerals and botanicals, and the additional active ingredients set out in Formulation Example 3.

An exemplary composition of a food or beverage product representing a composition of the invention is provided according to an embodiment. The composition is provided in a powder form, and designates a serving size or dosage of 13 g. The 13 g serving contains 5 g of an amino acid blend that includes: L-LEUCINE, L-LYSINE, L-ISOLEUCINE, L-VALINE, L-THREONINE, L-PROLINE, L-GLYCINE, L-ARGININE, L-HISTIDINE, L-CYSTINE, L-CYSTEINE, L-TYROSINE, L-PHENYLALANINE, L-METHIONINE, L-THEANINE, and L-TRYPTOPHAN.

The 13 g serving dosage also includes 1.8 g-2.5 g of a blend of additional components, which include medium chain triglycerides, Curcumin C3 Complex®, Curcumin C3 Reduct (Tetrahydrocurcumin standardized for 95% total curcuminoids), Silymarin, L-Carnitine, Trans-resveratrol, Taurine, sunflower lecithin, AlphaSize® 50WSP (Alpha-Glyceryl Phosphoryl Choline), Theracurmin®, CR-033P and Turmacin® Curcuma longa Extracts, PhosphatidylSerine 70% as SerinAid®, Ginko Biloba, Boron. In addition, there are Vitamins, which include, Vitamin C 50 mg, Thiamine B1 0.4 mg, Niacin (Vitamin B3-niacinamide) 20 mg, Vitamin B6 (pyridoxine) 0.4 mg, Folate (B9) 400 mg, Vitamin B12 6 mcg, Vitamin K (natural vitamin K2 as MenaQ7® Menaquinone-7) 90 mcg, Iron 0.3 mg, Magnesium 102 mg, and selenium (from SeleniumSeLECT® L-Selenomethionine) 73 mcg. The total carbohydrate amount in the composition is 8 g, which is contributed by the components mentioned, the total fat amount in the composition is 0.5 g, which is saturated fat and is contributed by the components mentioned. The composition is supplied in a container of about 390 g, and has a shelf life of over 3½ years.

The above formulations of nutritional supplements in the examples herein, including the Formulation Examples, preferably may be provided in a powder form that may be mixed with water or juice to form a suspension. The mixture of the formula and liquid (e.g., water or juice) may be ingested like any liquid drink or administered via feeding tube. Alternatively, the above formulations may be provided in the form of a tablet, or alternatively other solids or mediums, such as in a gel or gelatin, in a baked product, other food containing the composition, or other vehicle in which the formula may be supplied to an individual. According to some alternate embodiments, the formulations may be provided in a solution for feeding tube admission to an individual. Preferred excipients and diluents may be used to administer the formula, preferably to provide a stable medium, for systemic administration to an individual. Some preferred examples of excipients and diluents include starchy carbohydrates, low molecular weight non-starchy carbohydrates, suitable for tablets, capsules or other preparations, physiological saline or glucose solutions for feeding tube use.

The nutritional supplements described herein may be taken one or more times daily, where a daily dose may be one to four times.

The above nutritional supplement compositions may be produced in accordance with the amounts set out for the components of the formulas. For example, according to some embodiments, formulas may be provided that include EAAs, non-EAAs, vitamins, in combination with a complex carbohydrate, such as, for example, maltodextrin, as well as inactive ingredients (e.g., flavorings, storage enhancers, flowability agents, excipients, and the like). Preferred embodiments also may include one or more fatty substances, such as, for example, lecithin. The aforementioned compositions may be formulated in accordance with one or more of the weight percentages, or percentage ranges, for components that are specified above, and, preferred embodiments may include one or more substances, or all of the following, such as for example, turmeric (Turmacin), turmeric (95% curcuminoids), silymarin, resveratrol, Taurine, choline (choline-alFLOWsecrate (alpha GPC)), Curcuma longa extracts (Theracumin) 0.5%-3%, (Turmacin) 0.5%-3%, Curcumin C3 Complex (NLT 64:1 standardization ratio) 0.5% to 5%, Curcumin C3 Reduct (Tetrahydrocurcumin standardized for 95% total curcuminoids) 0.5%-0.3%, selenomethionine, magnesium citrate, ginko biloba.

The food and beverage compositions, according to preferred embodiments preferably include the amino acid blend which preferably also may be provided along with Vitamins, minerals and botanicals. According to a some preferred compositions, food and beverage compositions produced using the formulation may include amounts of branched-chain amino acids (BCAAs): isoleucine, leucine, and valine. According to preferred embodiments, the BCAAs may comprise up to about 45%, and preferably about 30-40% by weight of the active ingredients, when the active ingredients are amino acids, vitamins, minerals and botanicals (excluding maltodextrin, carbohydrates, fats (e.g., lecithin), flavorings, masking compounds, preservatives (e.g., citric acid, curcumin, turmeric). Vitamins, minerals and botanicals also are provided to form the composition, and may include those discussed herein. According to preferred compositions, the BCAAs of the formulation are present in a ratio (amongst themselves) by weight percent of about 50% leucine, and about 25% each of isoleucine and valine.

Preferred compositions include a cell-protective compound and a neuro-regenerative compound. These compositions may include, as the cell protective compound, phosphatidylserine (a phospholipid), and, as the neuro-regenerative compound, Vitamin B3 (niacinamide). According to a preferred composition, the ratio percent of phosphatidylserine to BCAAs is about 2%. Phosphatidylserine based on the weight percentage of active ingredients in the composition, preferably, is provided in the composition in an amount of less than 1%, and preferably about 0.6% to 0.9%, with a preferred amount being about 0.8%. The composition preferably includes a compound designated as a neuro-regenerative compound. According to preferred compositions, the neuro-regenerative compound comprises Vitamin B3 (niacinamide). According to a preferred composition, the ratio percent of Vitamin B3 (niacinamide) to BCAAs is about 0.7%. Vitamin B3 (niacinamide) based on the weight percentage of active ingredients in the composition, preferably, is provided in the composition in an amount of less than 1%, and preferably about 0.1% to 0.5%, with a preferred amount being about 0.25%.

The compositions of the invention preferably also include a turmeric component, which includes compounds that are derived from and/or extracted from, and/or composed of a turmeric based element. According to preferred compositions, the turmeric component comprises a mixture of: turmeric (95% curcuminoids) 0.5%-3%, Curcuma longa extracts (Theracumin) 0.5%-3%, (Turmacin) 0.5%-3%, Curcumin C3 Complex (NLT 64:1 standardization ratio) 1%-5%, Curcumin C3 Reduct (Tetrahydrocurcumin standardized for 95% total curcuminoids) 0.5% to 3%. According to preferred embodiments, the composition percentage of turmeric component in the formulation is about 3 to 7%, and preferably about 4% of the active ingredients (when the active ingredients are amino acids, vitamins, minerals and botanicals (excluding maltodextrin, carbohydrates, fats (e.g., lecithin), flavorings, masking compounds, preservatives (e.g., citric acid, curcumin, turmeric)). According to a preferred composition, the turmeric component in the formula comprises a turmeric mixture that includes the following relative weight percents of turmeric based elements: 16.7% Turmacin, 16.7% Curcuma Longa Extract-Theracurmin (NLT 33% Curcuminoids), 16.7% Curcumin C3 Reduct (Tetrahydrocurcumin standardized for 95% total curcuminoids), and 50% Curcumin C3 Complex (NLT 64:1 Standardization Ratio of Curcuminoids).

As with the compositions discussed herein, preferred compositions also include, in addition to the BCAAs, additional essential amino acids, including histidine, lysine, methionine, phenylalanine, threonine, and tryptophan, which preferably are present in the composition and comprise further proteinogenic amino acids in addition to the BCAAs.

According to preferred embodiments, the composition also includes additional amino acids. The additional amino acids present in preferred formulations include cysteine, histidine, phenylalanine, methionine, cystine, tyrosine, tryptophan, threonine and theanine. These amino acids may be present in amounts up to about 20% and preferably about 16.5% by weight of the active ingredients (when the active ingredients are amino acids, vitamins, minerals and botanicals (excluding maltodextrin, carbohydrates, fats (e.g., lecithin), flavorings, masking compounds, preservatives (e.g., citric acid, curcumin, turmeric)).

The following table, Table 4, includes an exemplary composition, where the active ingredients of the composition include amino acids, Vitamins, minerals and botanicals. The composition preferably may be ingested, provided in a suitable form for consumption. The exemplary formulation preferably may be taken with complex carbohydrates and/or fats. The food and beverage compositions may be produced using the formulation, and may be formulated to include carbohydrates and/or fats, preferably complex carbohydrates.

TABLE 4

L-LEUCINE
L-LYSINE
L-ISOLEUCINE
L-VALINE
L-THREONINE
L-PROLINE
L-GLYCINE
L-ARGININE
L-CYSTINE
L-HISTIDINE
L-PHENYLALANINE
L-METHIONINE
L-CYSTEINE
L-TYROSINE
L-TRYPTOPHAN
L-THEANINE
L-CARNITINE
THIAMINE (VITAMIN B1)
VITAMIN B3 (Niacinamide)
VITAMIN B6
VITAMIN B9 (Methylfolate)
VITAMIN B12 (METHYLCOBALAMIN)
VITAMIN C (ASCORBIC ACID)
Vitamin K2 MK7 as MenaQ7 Pure 2,000
PhosphatidylSerine 70% as SerinAid
AlphaSize ® 50WSP Alpha-Glyceryl Phosphoryl Choline
(ALPHA GPC)
TURMERIC (TURMACIN)
CURCUMA LONGA EXTRACT - Theracurmin (NLT 33%
Curcuminoids)
CURCUMIN C3 COMPLEX (NLT 64:1 Standardization Ratio of
Curcuminoids)
CURCUMIN C3 REDUCT (Tetrahydrocurcumin standardized
for 95% total curcuminoids)
GINGKO BILOBA 24%
SILYMARIN
TRANS-RESVERATROL
TAURINE
L-SELENOMETHIONINE as SeleniumSeLECT 39% Selenium
Boric Acid
MAGNESIUM CITRATE 16% (Nutrisol Brand)

In the above composition, the active ingredients are set forth. The above composition preferably may be formulated to include an amino acid mixture, which comprises 5 g of a 6 g composition. The composition may be a serving size of 6 g, of which about 5 g is amino acids, and a preferred dosage involves two servings of 6 g daily, or about 12 g.

In addition to the composition set forth in Table 4, above, the composition may be formulated with additional components, which preferably may include one or more of fats carbohydrates, preserving compounds, masking compounds, and flavorings. The composition may be formulated in any suitable ingestible medium, and may be administered as a powder, liquid, and may be consumed through drinking, eating, or dispensing through a feeding tube. According to a preferred composition, the formulation of Table 4 is provided with maltodextrin, and MCT, and lecithin, along with flavorings and masking agents. Table 5 below sets out a number of additional components, which, in the example below are balance ingredients. The balance ingredients preferably include:

TABLE 5

MCT Neobee 70% Powder
Sunflower Lecithin
ORGANIC TAPICOA MALTODEXTRIN DE 10
NATURAL MASKING FLAVOR
NATURAL CITRUS FLAVOR
Citric Acid
Stevia 97-99%
LuoHan Guo 50% Magrosides by UV
Faststir Xanthan EC
Flavoring

The above balance components preferably are added in suitable amounts, and may comprise an equal amount of the primary active ingredient amounts of the components of Table 5. For example, where the serving size of the active ingredients is about 6 g, the balance components preferably also are about 6 g, and more preferably, slightly greater, about 7 g. According to preferred embodiments, the MCT may be provided in an amount about equal to the amount of leucine. Preferred amounts by weight percent to the total composition including the active ingredients of Table 4, and including the components of Table 5, are about 5 to 10% MCT, 0.5 to 4% lecithin, 18 to 26% maltodextrin, about 10 to 20% masking and flavorings, about 2 to 3% citric acid and other texturizing compositions.

### Food or Beverage Product Example 1

According to a preferred formulation, a food product is provided having a composition that provides the amino acid blend (of TABLE A, above) in an amount of about 5 g, and a vitamin portion containing select vitamins listed in Formulation Example 1. Such food product could be a chocolate or nut butter snack bar, provided as an individually packaged food bar or multiple praline type chocolate balls packaged in a foil pack.

According to a preferred formulation, a food or beverage product is provided having a composition that provides the amino acid blend (of TABLE A, above) in an amount of about 5 g, and the vitamin portion containing the vitamins listed in Formulation Example 1, above, is about 0.1 to about 0.2 g, providing about 5.1 to 5.2 g of amino acids and vitamins. Preferably, the composition includes Vitamin B3 (niacinamide) in an amount of about 0.075 to 0.4% by weight of active ingredients, and about 0.2 to about 0.6 mg of the amino acid Vitamin mixture (of the 5.1 to 5.2 g). A binder vehicle is provided to present the amino acids and Vitamin mixture in a food product that may be ready to eat. The binder in this FOOD OR BEVERAGE PRODUCT EXAMPLE 1, is made from one or more of the following: eggs, wheat flour (or other flour, coconut, potato, sweet potato, chicory root, rice, and the like), cracker crumbs, oats or oatmeal, rice, milk, evaporated milk, coconut shavings, chocolate, cocoa, coffee, gelatin, guar gum, xanthan gum, psyllium husk, potato starch, ground flax, tapioca.

### Food or Beverage Product Example 2

According to a preferred formulation, a food product is provided having a composition that provides the amino acid blend (of TABLE A, above) in an amount of about 5 g, and a vitamin portion containing select vitamins and botanical ingredients listed in Formulation Example 1 as a meat substitute patty, such as would be consumed in an hamburger.

According to a preferred formulation, a food or beverage product is provided having a composition that provides the amino acid blend (of TABLE A, above) in an amount of about 5 g, and the vitamin portion containing the vitamins listed in Formulation Example 1, above, is about 0.1 to about 0.2 g, providing about 5.1 to 5.2 g of amino acids and vitamins. Preferably, the composition includes Vitamin B3 (niacinamide) in an amount of about 0.075 to 0.4% by weight of active ingredients, and about 0.2 to about 0.6 mg of the amino acid Vitamin mixture (of the 5.1 to 5.2 g). A binder vehicle is provided to present the amino acids and Vitamin mixture in a food product that may be ready to eat. The binder in this FOOD OR BEVERAGE PRODUCT EXAMPLE 1, is made from a nut butter, such as coconut butter, almond butter, peanut butter or similar. The nut butter preferably is provided in about 15 g.

### Food or Beverage Product Example 3

According to a preferred formulation, a food product is provided having a composition that provides the amino acid blend (of TABLE A, above) in an amount of about 5 g, and a vitamin portion containing select vitamins listed in Formulation Example 1 as a brownie or individually wrapped cake.

This is the components of Food or Beverage Formulation Example 2, and including egg white (about 0.5 grams of dried egg white) and an additional sugar, the sugar being honey (about 1 g). The food product is supplied in a paste or butter type form that may be stored in individual sealed packets (foil and/or plastic), and may be provided in a spreadable form. Alternatively, the product may be dried to form a crumble or bar.

### Food or Beverage Product Example 4

This is the components of Food or Beverage Formulation Example 2, and including an additional sugar, the sugar being honey (about 1 g), and includes a mixture of a flour product (e.g., acacia fiber, chicory fiber potato fiber). Organic Tapicoa Maltodextrin DE 10 is added. The food product is dried or baked to produce a dry product that may be stored in individual sealed packets (foil and/or plastic) or other container.

According to some embodiments, the above amino acid compositions preferably are provided in the final food product to have the composition ratios of the amino acids. For example, where a binding agent or other component is added, the amino acid contribution of that substance (e.g., the binding agent and/or the component) is counted in the amino acid content. According to these embodiments, the formulations therefore preserve the balance of the ratios, in accordance with the formulation embodiments set forth above. The balance of the ratios may be essential amino acids (EAA) to non-EAAs, or may be branched chain amino acids, or the amino acid or acid amounts, as well as amino acid ratios to other components, such as Vitamins and/or minerals, and/or botanicals, carbohydrates, and other components. The additional presence of Vitamins, minerals, and botanicals also is benefitted by the synergistic effects which, according to some embodiments, correlate with the amino acids and the amounts or ratios (presence of the amount of one or more amino acids to one or more other amino acids).

Although throughout some brand names may be indicated according to preferred embodiments, which may be referred to by brand or trademark, alternatively, the source of the compound or substance may be supplied under another brand or by a different source (such as, for example, under a generic name, or provided under another mark).

The formulations including those set forth in the examples provided herein may comprise or be used to provide a sustainable food product, that provides protein needs for individuals. The compositions may be used to provide sustenance for individuals and populations in areas where foods that provide suitable nutrients are scarce due to economic, environmental, weather or other conditions. The compositions provide nutrients and synergistic ratios and combinations of amino acids, including essential and non-essentials amino acids, along with Vitamins, and nutrients including botanicals, which are provided as a protein substitute. Embodiments may be provided in the form of products that may be stored, packaged and delivered for consumption.

What is claimed:

1. A food or beverage product containing:
a) amino acids, comprising:
i) EAAs selected from the group consisting of: isoleucine, leucine, valine, histidine, lysine, methionine, phenylalanine, threonine, and tryptophan;
ii) non-EAAs selected from the group consisting of: arginine, cysteine, cystine, glycine, proline, and tyrosine;
b) one or more of a cell protective compound and a neuro-regenerative compound.

2. The food or beverage product of claim 1, including binder material comprising one or more plant based materials.

3. The food product of claim 1, including both the cell protective compound and the neuro-regenerative compound.

4. The food or beverage product of claim 1, wherein said cell protective compound comprises phosphatidylserine.

5. The food product of claim 1, wherein said neuro-regenerative compound comprises Vitamin B3.

6. The food product of claim 3, wherein said cell protective compound comprises phosphatidylserine, and wherein said neuro-regenerative compound comprises Vitamin B3.

7. The food or beverage product of claim 1, including Vitamin K2 MK7 and boric acid.

8. The food or beverage product of claim 1,
wherein the EAAs are provided to comprise about 74% to 77% by weight of the total amino acids of the food product,
wherein the non-EAAs comprise about 23% to 26% by weight of the total amino acids of the food product.

9. The food or beverage product of claim 3,
wherein the EAAs are provided to comprise about 74% to 77% by weight of the total amino acids of the food or beverage product,
wherein the non-EAAs comprise about 23% to 26% by weight of the total amino acids of the food or beverage product.

10. The food or beverage product of claim 9, wherein the amino acid composition remains the same after the addition of the binder material.

11. The food product of claim 9, wherein the amino acids are formed with the binder material.

12. The food product of claim 11, wherein the binder material comprises a carbohydrate mixture that is a gellable component, wherein said gellable component is formed to include said amino acids, and wherein one or more of a flavorant and texturizer is added to form the food or beverage product.

13. The food or beverage product of claim 12, wherein said texturizer comprises an aggregate of fibers of the gellable component that includes said amino acids.

14. The food or beverage product of claim 13, wherein said gellable component fibers comprise said amino acids, and wherein said amino acids are present in said gellable component, in said percentage of said EAA to said non-EAA.

15. The food or beverage product of claim 13, wherein said flavorant comprises a natural flavoring from one or more of fruits, vegetables, or other plants.

16. The food or beverage product of any one of claims 1-11, wherein the food or beverage product provides bioavailable nutrients, the product further including:
a complex carbohydrate; and
magnesium citrate.

17. The food or beverage product of claim 16, wherein the complex carbohydrate comprises maltodextrin.

18. The food or beverage product of claim 16, wherein the composition includes at least a quantity of each EAA of paragraph a), and at least a quantity of each non-EAA of paragraph b).

19. The food or beverage product of claim 16, further including a fatty substance.

20. The food or beverage product of claim 19, wherein said fatty substance comprises lecithin.

21. The food or beverage product of claim 16, including, as active ingredients, turmeric (turmacin), turmeric (95% curcuminoids), silymarin, resveratrol, taurine, choline (choline-alFLOWsecrate (alpha GPC)), Curcuma longa extract (theracumin), curcumin c3 complex (NLT 64:1 standardization ratio), Curcumin C3 Reduct (Tetrahydrocurcumin standardized for 95% total curcuminoids), Vitamin K2 MK7, Vitamin C (ascorbic acid), thiamine (Vitamin B1), Vitamin B3 (Niacinamide), Vitamin B12 (methylcobalamin), Vitamin B6, Vitamin B9 (Methylfolate), phosphatidylserine, boric acid, selenomethionine, magnesium citrate, ginko biloba, MCT Neobee® 50%-70% powder, and a fatty substance, wherein the complex carbohydrate comprises maltodextrin;
and wherein said amino acids comprise active ingredients, and
wherein the EAAs are provided to comprise percent by weight of the active ingredients of the composition of about 32 to 34%; and
wherein the non-EAAs are provided to comprise percent by weight of the active ingredients of the composition of about 9 to 11%;
and wherein said active ingredients and said binder form said food or beverage product.

22. The food or beverage product of claim 19, wherein the fatty substance comprises lecithin (e.g., sunflower lecithin).

23. The food or beverage product of claim 1, including Vitamin K2 MK7 (MenaQ7 Pure 2000), Vitamin C (ascorbic acid), thiamine (Vitamin B1), Vitamin B3 (Niacinamide), Vitamin B12 (methylcobalamin), Vitamin B6, and Vitamin B9 (Methylfolate), each in amounts of less than 1% by weight of the active ingredients, and wherein Vitamin C is provided in an amount at least 10 times greater by weight percent of active ingredients, than each amount of each other of said vitamins: thiamine (Vitamin B1), Vitamin B12 (methylcobalamin), Vitamin B6, and Vitamin B9 (Methylfolate), and wherein Vitamin K2 MK7 (MenaQ7 Pure 2000) is provided at least 10 times greater by weight percent of active ingredients, than each amount of each other of said vitamins: thiamine (Vitamin B1), Vitamin B12 (methylcobalamin), Vitamin B6, and Vitamin B9 (Methylfolate), and)), and wherein Vitamin B3 is provided at least 10 times greater by weight percent of active ingredients, than each amount of each other of said vitamins: thiamine (Vitamin B1), Vitamin B12 (methylcobalamin), Vitamin B6, and Vitamin B9 (Methylfolate).

24. The food or beverage product of claim 21, wherein maltodextrin is provided at a ratio of 1:1 to the essential amino acids in the composition.

25. The food or beverage product of claim 22, wherein maltodextrin is present in an amount that is about 75% by weight of the amount of total amino acids in the composition.

26. The food or beverage product of claim 25, wherein the total amino acids are present in the composition in an amount of about 40 to 46% by weight of the active ingredients, when maltodextrin, MCT and lecithin are present as active ingredients.

27. The food or beverage product of claim 23, wherein the total amino acids are present in the composition in an amount of about 70 to 85% by weight of the active ingredients, when the active ingredients are amino acids, vitamins, minerals and botanicals.

28. A food or beverage product for providing bioavailable nutrients, the food or beverage product comprising:
a) a binder material comprising one or more plant based materials;
b) EAAs selected from the group consisting of: Isoleucine, Leucine, Valine, Histidine, Lysine, Methionine, Phenylalanine, Threonine, and Tryptophan, Histidine, Lysine, Methionine, Phenylalanine, Threonine, and Tryptophan;
c) Non-EAA selected from the group consisting of: Arginine, Cysteine, Cystine, Glycine, Proline, and Tyrosine;
d) maltodextrin;
e) lecithin, and
f) magnesium citrate;
g) wherein the EAAs are provided to comprise percent by weight of the active ingredients of the composition are present in a major amount relative to the non-EAAs; and
h) wherein the composition includes at least a quantity of each EAA of paragraph b), and at least a quantity of each non-EAA of paragraph c); and
i) one or more inactive ingredients comprising one or more flavoring substances, and
j) as active ingredients, an amount of each of the following: Carnitine, Curcuma longa extracts as Turmacin®, Curcumin C3 Complex® (NLT 64:1 standardization ratio), Curcumin C3 Reduct (Tetrahydrocurcumin standardized for 95% total curcuminoids), turmeric (95% curcuminoids), curcurmin, TheraCurmin®, LongVida®, silymarin, trans-resveratrol, Taurine, Alpha-Glyceryl Phosphoryl Choline (as choline-alFLOWsecrate®, AlphaSize® or other forms of alpha GPC), PhosphatidylSerine (preferably SerinAid®), Vitamin K2 MK7 (preferably MenaQ7® MK7 form of vitamin K2), Vitamin C (ascorbic acid), thiamine (Vitamin B1), Vitamin B3 (Niacinamide) Vitamin B12 (methylcobalamin), Vitamin B6, Vitamin B9 (Methylfolate), selenomethionine SeleniumSeLECT®, a form of boron, such as boric acid and/or boron citrate, boron glycinate, and/or boron aspartate, a magnesium source (such as magnesium citrate), ginko biloba, complex carbohydrate (e.g., maltodextrin), medium chain triglycerides, (e.g., MCT Neobee® 50-70% powder), a fatty substance, such as lecithin (e.g., sunflower or canola lecithin).

29. The food or beverage product of claim 1, including a carbohydrate, wherein said carbohydrate comprises a mixture of simple and complex sugar producing carbohydrates selected from the group consisting of coconut palm sugar and fructose, maltodextrin and resistant starches, and mixtures thereof.

30. The food or beverage product of claim 29, wherein said resistant starches are selected from the group consisting of green banana flower, chicory root and mixtures thereof.

31. The food or beverage product of claim 1, wherein the EAAs and non-EAAs are provided in a single serving size that contains 5 g of a mixture of the amino acids: isoleucine, leucine, valine, histidine, lysine, methionine, phenylalanine, threonine, tryptophan, arginine, cysteine, cystine, glycine, proline, and tyrosine.

32. The food or beverage product of claim 31, wherein of the 5 g mixture of the amino acids: isoleucine, leucine, valine, histidine, lysine, methionine, phenylalanine, threonine, tryptophan, arginine, cysteine, cystine, glycine, proline, and tyrosine, about 3.75 g of the 5 g amino acid mixture consists of: isoleucine, leucine, valine, histidine, lysine, methionine, phenylalanine, threonine, tryptophan, and wherein about 1.25 g of the 5 g amino acid mixture consists of: arginine, cysteine, cystine, glycine, proline, and tyrosine.

33. The food or beverage product of claim 1, wherein the composition includes at least a quantity of each EAA of paragraph a), and at least a quantity of each non-EAA of paragraph b), and Vitamin B.

34. The food or beverage product of claim 1, wherein the composition includes at least a quantity of each EAA of paragraph a), and at least a quantity of each non-EAA of paragraph b), and a turmeric composition.

35. The food or beverage product of claim 34, wherein the turmeric composition comprises turmeric compositions selected from the group consisting of: Turmacin, Curcuma longa Extract-Theracurmin (NLT 33% Curcuminoids), Curcumin C3 Complex (NLT 64:1 Standardization Ratio of Curcuminoids), and Curcumin C3 Reduct (Tetrahydrocurcumin standardized for 95% total curcuminoids).

36. The food or beverage product of claim 35, wherein the turmeric components are provided in the following weight percentages relative to each of the turmeric components in the product: 16.7% Turmacin, 16.7% Curcuma longa Extract-Theracurmin (NLT 33% Curcuminoids), and 50% Curcumin C3 Complex (NLT 64:1 Standardization Ratio of Curcuminoids), 16.7% Curcumin C3 Reduct (Tetrahydrocurcumin standardized for 95% total curcuminoids).
